(19) United States
(12) Patent Application Publication
(10) Pub. No.: US 2023/0048366 A1
(43) Pub. Date: Feb. 16, 2023

(54) COMBINATION THERAPY FOR TREATING OR PREVENTING CANCER

(71) Applicant: 4D Pharma Research Limited, Aberdeen (GB)

(72) Inventor: Alexander STEVENSON, Leeds (GB)

(21) Appl. No.: 17/936,963

(22) Filed: Sep. 30, 2022

Related U.S. Application Data
(63) Continuation of application No. 16/932,052, filed on Jul. 17, 2020.
Application No. 16/932,052 is a continuation of application No. PCT/GB2019/050141, filed on Jan. 18, 2019.

(30) Foreign Application Priority Data
Jan. 19, 2018 (GB) 1800927.4
Jan. 30, 2018 (GB) 1801502.4
Apr. 10, 2018 (GB) 1805941.0
Apr. 23, 2018 (GB) 1806572.2
May 25, 2018 (GB) 1808628.0

(51) Int. Cl. A61K 35/744; A61P 35/00; A61K 9/00; A61K 39/395

(52) U.S. Cl. CPC A61K 35/744; A61P 35/00; A61K 9/0019; A61K 9/0053; A61K 39/3955; A61K 2039/545

(57) ABSTRACT

The invention provides a combination therapy comprising a bacterial strain for treating or preventing cancer.

### CROSS REFERENCE

This application is a continuation of U.S. application Ser. No. 16/932,052, filed on Jul. 17, 2020, which is a continuation of International Application No. PCT/GB2019/050141, filed on Jan. 18, 2019, which claims priority to GB Application No. 1800927.4, filed on Jan. 19, 2018, GB Application No. 1801502.4, filed on Jan. 30, 2018, GB Application No. 1805941.0, filed on Apr. 10, 2018, GB Application No. 1806572.2, filed on Apr. 23, 2018, and GB Application No. 1808628.0, filed on May 25, 2018, each of which is herein incorporated by reference in its entirety.

### SEQUENCE LISTING

The instant application contains a Sequence Listing which has been submitted electronically in Extendible Markup Language (XML) format and is hereby incorporated by reference in its entirety. Said XML copy, created on Sep. 23, 2022, is named 56708_735_302_SL.xml and is 5,744 bytes in size.

### TECHNICAL FIELD

This invention is in the field of a combination therapy for treating or preventing cancer: a combination of a composition comprising a bacterial strain and pembrolizumab for treating or preventing cancer.

### BACKGROUND TO THE INVENTION

The human intestine is thought to be sterile in utero, but it is exposed to a large variety of maternal and environmental microbes immediately after birth. Thereafter, a dynamic period of microbial colonization and succession occurs, which is influenced by factors such as delivery mode, environment, diet and host genotype, all of which impact upon the composition of the gut microbiota, particularly during early life. Subsequently, the microbiota stabilizes and becomes adult-like [1]. The human gut microbiota contains more than 500-1000 different phylotypes belonging essentially to two major bacterial divisions, the Bacteroidetes and the Firmicutes [2]. The successful symbiotic relationships arising from bacterial colonization of the human gut have yielded a wide variety of metabolic, structural, protective and other beneficial functions. The enhanced metabolic activities of the colonized gut ensure that otherwise indigestible dietary components are degraded with release of by-products providing an important nutrient source for the host. Similarly, the immunological importance of the gut microbiota is well-recognized and is exemplified in germfree animals which have an impaired immune system that is functionally reconstituted following the introduction of commensal bacteria [3-5].

Dramatic changes in microbiota composition have been documented in gastrointestinal disorders such as inflammatory bowel disease (IBD). For example, the levels of Clostridium cluster XIVa bacteria are reduced in IBD patients whilst numbers of E. coli are increased, suggesting a shift in the balance of symbionts and pathobionts within the gut [6-9]. Interestingly, this microbial dysbiosis is also associated with imbalances in T effector cell populations.

In recognition of the potential positive effect that certain bacterial strains may have on the animal gut, various strains have been proposed for use in the treatment of various diseases (see, for example, [10-13]). Also, certain strains, including mostly Lactobacillus and Bifidobacterium strains, have been proposed for use in treating various inflammatory and autoimmune diseases that are not directly linked to the intestines (see [14] and [15] for reviews). However, the relationship between different diseases and different bacterial strains, and the precise effects of particular bacterial strains on the gut and at a systemic level and on any particular types of diseases, are poorly characterised. For example, certain Enterococcus species have been implicated in causing cancer [16]. In contrast, bacterial strains of the species Enterococcus gallinarum have also been disclosed for use in treating and preventing cancer [54].

Due to the diverse nature of cancer, various treatment modalities are being developed in order to treat different patient groups. One treatment modality that has proved effective is the use of Immune Checkpoint Inhibitors (ICIs). ICIs are compounds that inhibit a cancer cell's ability to prevent the host's immune cells from attacking cancer cells. ICIs may be, for instance, therapeutic antibodies that have been developed against the interaction between the transmembrane receptor programmed cell death 1 protein (referred to as PDCD1, PD-1, PD1, or CD279) and its ligand, PD-1 ligand 1 (referred to as PD-L1, PDL1 or CD274). An example for such an antibody is Pembrolizumab, which targets PD-1 (marketed by Merck under the commercial name KEYTRUDA®).

Although treatment of cancer patients with an ICI, when effective, can result in long lasting and significant clinical effects, there is still a significant percentage of patients that are non-responsive or only partially responsive to ICI treatment. There is therefore a requirement in the art for new and improved treatment modalities to prevent and treat cancer, and in particular treatment modalities which may improve the effect of pembrolizumab treatment.

### SUMMARY OF THE INVENTION

The present invention relates to novel combination therapies for treating and preventing cancer. In particular, the present invention relates to improved therapies in which sequential and/or partially parallel administration of a bacterial strain of the species Enterococcus gallinarum and pembrolizumab results in a more effective treatment of cancer than treatment with the bacterial strain or pembrolizumab alone.

Compositions comprising a bacterial strain of the species Enterococcus gallinarum are effective in therapy in general, and in treating or preventing cancer in particular, as presented herein below and in [54]. The present invention is further based in part on the unexpected effect achieved upon administration of both pembrolizumab and a composition comprising a bacterial strain of the species Enterococcus gallinarum. As used herein, the terms “the combination of the invention”, “the therapeutic combination of the invention” and “the therapeutic combination” may be used interchangeably and refer to a therapeutic combination of: (a) a composition comprising a bacterial strain of the species Enterococcus gallinarum; and (b) pembrolizumab. It is to be understood that the term “combination” in the context of the therapeutic combination does not refer to components (a) and (b) of the combination necessarily being in the same composition and/or administered at the same time. According to preferred embodiments, (a) and (b) of the therapeutic combination are in separate compositions. According to some embodiments, provided herein is the combination of the invention for use in a method of treating or preventing cancer in a subject. According to some embodiments, provided herein is a method for treating or preventing cancer in a subject, comprising administering the therapeutic combination of the invention to the subject.

According to some embodiments, administration of the bacterial composition in the context of the therapeutic combination enables treatment of cancer patients who were non-responsive or who showed insufficient response to treatment with an immune checkpoint inhibitor that was administered without the bacterial composition. According to some embodiments, the patients who are non-responsive or partial responders to ICI therapy may be ICI naïve (i.e. they have not previously received ICI therapy) or they may have become non-responders or partial responders following previously successful administration of ICIs.

Without wishing to be bound by theory or mechanism, this effect might be through modulation of mediators that improve the efficiency of pembrolizumab, such as through an increase in tumour-infiltrating CD8⁺ T-cells or an increase in the ratio of tumour-infiltrating CD8⁺ T-cells to FoxP3+ cells.

According to one aspect, provided herein is a therapeutic combination for use in a method of treating or preventing cancer in a subject, wherein said therapeutic combination comprises:
(a) a composition comprising a bacterial strain of the species Enterococcus gallinarum; and
(b) pembrolizumab.

According to some embodiments, provided herein is a composition comprising a bacterial strain of the species Enterococcus gallinarum for use in a method of treating or preventing cancer in a subject, wherein said composition is used in combination with pembrolizumab.

According to some embodiments, provided herein is a first composition comprising a bacterial strain of the species Enterococcus gallinarum for use in combination with a second composition comprising pembrolizumab, for use in a method of treating or preventing cancer in a subject, optionally wherein said first composition is administered prior to first administration of said second composition and/or in parallel to the administration of the second composition, optionally wherein the subject was non-responsive to a prior treatment using an immune checkpoint inhibitor alone.

According to another aspect, provided herein is a method of treating or preventing cancer in a subject in need thereof (referred to herein also as “the method of the invention”), the method comprising: (a) administering to the subject a composition comprising a bacterial strain of the species Enterococcus gallinarum; and (b) administering to the subject pembrolizumab.

According to another aspect, provided herein is a kit comprising: (a) a composition comprising a bacterial strain of the species Enterococcus gallinarum; and (b) a composition comprising pembrolizumab.

According to some embodiments, cancer is selected from the group consisting of: breast cancer, lung cancer, colon cancer, kidney cancer, liver cancer, lymphoma (such as non-Hodgkin's lymphoma), hepatoma and neuroendocrine cancer. According to some embodiments, the therapeutic combination is for use in a method of treating or preventing lung cancer, breast cancer, kidney cancer, liver cancer, lymphoma, hepatoma, neuroendocrine cancer or colon cancer. According to some embodiments, cancer is selected from the group consisting of: melanoma, non-small cell lung carcinoma, bladder cancer and head-and-neck cancer. In certain embodiments, the therapeutic combination or the method of the invention is for use in reducing tumour size or preventing tumour growth in the treatment of cancer. According to some embodiments, the therapeutic combination or the method of the invention is for use in at least one of reducing tumour size, reducing tumour growth, preventing metastasis or preventing angiogenesis.

According to some embodiments, the terms “the composition”, “the bacterial composition” and “the composition of the invention” may be used interchangeably and refer to the composition included in the therapeutic combination of the invention, which comprises a bacterial strain of the species Enterococcus gallinarum. According to some embodiments, the composition comprising a bacterial strain of the species Enterococcus gallinarum does not contain bacteria from any other species or comprises only de minimis or biologically irrelevant amounts of bacteria from another species. According to some embodiments, closely related strains of Enterococcus gallinarum may also be used as part of the therapeutic combination, such as bacterial strains that have a 16s rRNA sequence that is at least 95%, 96%, 97%, 98%, 99%, 99.5% or 99.9% identical to the 16s rRNA sequence of a bacterial strain of Enterococcus gallinarum. Preferably, the bacterial strain has a 16s rRNA sequence that is at least 95%, 96%, 97%, 98%, 99%, 99.5% or 99.9% identical to SEQ ID NO:1 or 2. Preferably, the sequence identity is to SEQ ID NO:2. Preferably, the bacterial strain for use in the therapeutic combination of the invention has the 16s rRNA sequence represented by SEQ ID NO:2.

Accordingly, the therapeutic combination of the invention may comprise a composition comprising a bacterial strain that has a 16s rRNA sequence that is at least 95% identical to the 16s rRNA sequence of a bacterial strain of Enterococcus gallinarum, optionally to SEQ ID NO: 2, for use in a method of treating or preventing cancer. Enterococcus gallinarum In some embodiments, the bacterial strain in the composition is not of Enterococcus gallinarum, but is a closely related strain.

In certain embodiments, the composition of the invention is for oral administration. Oral administration of the strains of the invention can be effective for treating cancer, in particular when administered as part of the therapeutic combination of the invention. Also, oral administration is convenient for patients and practitioners and allows delivery to and/or partial or total colonisation of the intestine. According to some embodiments, the pembrolizumab used as part of the therapeutic combination of the invention is administered intravenously. According to some embodiments, each of the bacterial composition and the pembrolizumab of the therapeutic combination are present in a separate composition, each possibly comprising a carrier and/or an excipient suitable for its mode of administration. In certain embodiments, the composition of the invention comprises one or more pharmaceutically acceptable excipients or carriers. In certain embodiments, the pembrolizumab is in a composition comprising one or more pharmaceutically acceptable excipients or carriers.

In certain embodiments, the bacterial composition of the invention comprises a bacterial strain that has been lyophilised. Lyophilisation is an effective and convenient technique for preparing stable compositions that allow delivery of bacteria. According to some embodiments, the bacterial strain in the composition is capable of partially or totally colonising the intestine.

In certain embodiments, the bacterial composition is comprised in a food product. In certain embodiments, the bacterial composition is comprised in a vaccine.

According to some embodiments, the bacterial composition comprises a single strain of Enterococcus gallinarum. According to some embodiments, the bacterial composition comprises the Enterococcus gallinarum bacterial strain as part of a microbial consortium. Preferably, the bacterial composition comprises the Enterococcus gallinarum strain deposited under accession number NCIMB 42488.

According to some embodiments of the method of the invention, the bacterial composition is administered to the subject prior to a first administration of pembrolizumab to the subject. According to some embodiments of the method of the invention, the bacterial composition is administered to the subject for at least one, two, three or four weeks prior to first administration of pembrolizumab. It is to be understood that in the context of the method of the invention, the first administration of pembrolizumab refers to a first administration as part of the therapeutic combination of the invention. Prior to administration of the therapeutic combination of the invention the subject might have been administered with pembrolizumab without the bacterial composition of the invention being administered during/before administration of pembrolizumab. According to some embodiments, at least one, two, three or four weeks passed between administration of the therapeutic combination of the invention and prior administration of pembrolizumab alone or the bacterial composition alone.

According to some embodiments of the method of the invention, the bacterial composition is administered to the subject at least partially in parallel to administration of pembrolizumab to the subject. In the context of administration times of the bacterial composition and pembrolizumab, administration at least partially in parallel refers to administrations which may overlap completely (for example, administration of both components over a course of 12 months) or partially (for example, administration of one component over a course of 12 months and administration of the second component over a course of 8 months, which may overlap completely or partially with the 12 month period). It is to be understood that parallel administration of both components does not mean that both components are necessarily administered using the same dosage regime. According to some embodiments of the method of the invention, the bacterial composition is administered to the subject prior to first administration of pembrolizumab and/or at least partially in parallel to administration of pembrolizumab to said subject. According to certain embodiments, the bacterial composition is administered to the subject for at least one, two, three or four weeks prior to first administration of pembrolizumab, followed by administration of the bacterial composition and pembrolizumab at least partially in parallel for at least two, four or six weeks.

According to some embodiments, the bacterial strain of the species Enterococcus gallinarum and pembrolizumab are in separate compositions, preferably wherein the bacterial composition is formulated for oral administration whereas pembrolizumab is in a formulation formulated for intravenous administration.

According to some embodiments, the therapeutic combination of the invention is for treating or preventing cancer in a subject who was non-responsive to a prior treatment using an immune checkpoint inhibitor alone. As used herein, a subject who is non-responsive to treatment with an immune checkpoint inhibitor relates to a subject who is non-responsive according to the RECIST (Response Evaluation Criteria In Solid Tumours) criteria or according to the irRECIST (immune-related Response Evaluation Criteria In Solid Tumours) criteria.

According to some embodiments, the therapeutic combination of the invention is for treating or preventing cancer in a subject in which pembrolizumab or the bacterial composition alone cannot provide effective treatment or prevention of cancer in the subject. According to some embodiments, an effective treatment of cancer in a subject comprises at least one of reducing tumour size, reducing tumour growth and/or preventing metastasis to an extent which will result in complete or partial remission of the cancer in the subject.

According to some embodiments, the therapeutic combination of the invention is capable of reducing tumour size and/or reducing tumour growth and/or preventing metastasis and/or preventing angiogenesis to a higher extent than pembrolizumab or the bacterial composition alone.

According to some embodiments, the therapeutic combination of the invention is for treating cancer in a subject, such that there is complete remission of cancer in the subject, preferably in a shorter time frame than that achieved using treatment with pembrolizumab or the bacterial composition alone.

The invention also provides a composition comprising Pembrolizumab, for use in a method of treating or preventing cancer in a subject that had previously received administration of a composition comprising a bacterial strain of the species Enterococcus gallinarum, preferably the strain deposited under accession number NCIMB 42488.

The invention also provides a composition comprising a bacterial strain of the species Enterococcus gallinarum, preferably the strain deposited under accession number NCIMB 42488, for use in a method of treating or preventing cancer in a subject diagnosed as requiring treatment with Pembrolizumab.

### BRIEF DESCRIPTION OF DRAWINGS

FIG. 1A: Mouse model of breast cancer-tumor volume.

FIG. 1B: Upper panel: Area of necrosis in EMT6 tumours (Untreated n=6, Vehicle n=6, MRx0518 n=8). Lower panel: Percentage of dividing cells in EMT6 tumours. P=0.019 (Untreated n=4, total number cells counted=37201, Vehicle n=6, total number of cells counted=64297, MRx0518 n=6, total number cells counted=33539).

FIG. 1C: Mouse model of breast cancer-infiltrating immune cells. Scatter plots represent cell counts of different immune markers from individual animals from each treatment group.

FIG. 1D: Mouse model of breast cancer-Cytokine production in tumour lysates. Columns represent the mean pg/mL of total protein from each treatment group. *p<0.05 between groups using one-way ANOVA followed by Dunnett's multiple comparisons test.

FIG. 1E: Mouse model of breast cancer-Cytokine production in blood plasma. Columns represent the mean pg/mL from each treatment group (+/−SEM).

FIG. 1F: Representative images of ileum cryosections from vehicle, MRx0518 and CTLA-4-treated mice immuno-labelled with antibodies against CD8α (lower panels) and counter-stained with DAPI (upper panels).

FIG. 1G: Plot quantifying animal study subsets with more than 3 CD8α+ cells per field taken from the ileum crypt region of mice treated with vehicle, MRx0518 or CTLA-4.

FIG. 2: Mouse model of lung cancer-tumour volume.

FIG. 3A: Mouse model of liver cancer-liver weight.

FIG. 3B: Mouse model of kidney cancer-tumour volume.

FIG. 4A: Cytokine levels (pg/ml) in immature dendritic cells (No bacteria).

FIG. 4B: Cytokine levels (pg/ml) in immature dendritic cells after the addition of LPS.

FIG. 4C: Cytokine levels (pg/ml) in immature dendritic cells after the addition of MRX518.

FIG. 4D: Cytokine levels (pg/ml) in immature dendritic cells after the addition of MRX518 and LPS.

FIG. 5A: Cytokine levels in THP-1 cells (No bacteria).

FIG. 5B: Cytokine levels in THP-1 cells after addition of bacterial sediment.

FIG. 5C: Cytokine levels in THP-1 cells after the addition of MRX518 alone or in combination with LPS.

FIG. 6: Bar graph depicting percentage of proliferating CD8+ cells following various treatments (NCD—No Cell Division, 1RCD—One Cell Division, 2RCD—Two Cell Divisions, 3RCD—Three Cell Divisions, 4RCD—Four Cell Divisions).

FIG. 7A: A schematic representation of the treatment schedule of the different groups used in Example 6 described herein below.

FIG. 7B: Mean tumour volume in mice bearing a tumour formed by EMT-6 cells. The mice were either untreated or treated with a YCFA vehicle (Vehicle), MRx518 bacteria in YCFA medium (MRx518), an anti-PD1 antibody and YCFA medium (Anti-PD1), an anti-CTLA-4 antibody and YCFA medium (Anti-CTLA-4), a combination of MRx518 and the anti-PD1 antibody or a combination of MRx518 and the anti-CTLA-4 antibody.

### DISCLOSURE OF THE INVENTION

Bacterial Strains

The compositions of the invention comprise a bacterial strain of the species Enterococcus gallinarum. The examples demonstrate that a therapeutic combination comprising bacteria of this species is useful for treating or preventing cancer.

According to some embodiments, provided herein is a therapeutic combination for use in a method of treating or preventing cancer in a subject, wherein said therapeutic combination comprises:
(a) a composition comprising a bacterial strain of the species Enterococcus gallinarum; and
(b) pembrolizumab

According to some embodiments, a composition comprising a bacterial strain that has a 16s rRNA sequence that is at least 95% identical to the 16s rRNA sequence of a bacterial strain of Enterococcus gallinarum may be used in the therapeutic combination and method of the present invention. According to certain embodiments, the invention also provides a composition comprising a bacterial strain that has a 16s rRNA sequence that is at least 95% identical to SEQ ID NO: 2 for use in treating or preventing cancer in combination with pembrolizumab. In some embodiments, the bacterial strain in the composition is not of Enterococcus gallinarum, but is a closely related strain.

In certain embodiments, the composition of the invention comprises a bacterial strain that has a 16s rRNA sequence that is at least 95% identical to SEQ ID NO: 2, for example which is a Enterococcus gallinarum, and does not contain any other bacterial genus. In certain embodiments, the composition of the invention comprises a single strain of a bacterial strain that has a 16s rRNA sequence that is at least 95% identical to SEQ ID NO: 2, for example, which is an Enterococcus gallinarum, and does not contain any other bacterial strain or species.

Enterococcus gallinarum forms coccoid cells, mostly in pairs or short chains. It is nonmotile and colonies on blood agar or nutrient agar are circular and smooth. Enterococcus gallinarum reacts with Lancefield group D antisera. The type strain of Enterococcus gallinarum is F871276=PB21=ATCC 49573=CCUG 18658=CIP 103013=JCM 8728=LMG 13129=NBRC 100675=NCIMB 702313 (formerly NCDO 2313)=NCTC 12359 [17]. The GenBank accession number for a 16S rRNA gene sequence of Enterococcus gallinarum is AF039900 (disclosed herein as SEQ ID NO:1). An exemplary Enterococcus gallinarum strain is described in [17].

All microorganism deposits were made under the terms of the Budapest Treaty and thus viability of the deposit is assured. Maintenance of a viable culture is assured for 30 years from the date of deposit. During the pendency of the application, access to the deposit will be afforded to one determined by the Commissioner of the United States Patent and Trademark Office to be entitled thereto. All restrictions on the availability to the public of the deposited microorganisms will be irrevocably removed upon the granting of a patent for this application. The deposit will be maintained for a term of at least thirty (30) years from the date of the deposit or for the enforceable life of the patent or for a period of at least five (5) years after the most recent request for the furnishing of a sample of the deposited material, whichever is longest. The deposit will be replaced should it become necessary due to inviability, contamination or loss of capability to function in the manner described in the specification.

The Enterococcus gallinarum bacterium deposited under accession number NCIMB 42488 was tested in the Examples and is also referred to herein as strain MRX518. References to MRX518 and MRx0518 are used interchangeably. A 16S rRNA sequence for the MRX518 strain that was tested is provided in SEQ ID NO:2. Strain MRX518 was deposited with the international depositary authority NCIMB, Ltd. (Ferguson Building, Aberdeen, AB21 9YA, Scotland) by 4D Pharma Research Ltd. (Life Sciences Innovation Building, Aberdeen, AB25 2ZS, Scotland) on 16 Nov. 2015 as “Enterococcus sp” and was assigned accession number NCIMB 42488.

The genome of strain MRX518 comprises a chromosome and plasmid. A chromosome sequence for strain MRX518 is provided in SEQ ID NO:3 of WO2017/085520. A plasmid sequence for strain MRX518 is provided in SEQ ID NO:4 of WO2017/085520. These sequences were generated using the PacBio RS II platform.

Bacterial strains closely related to the strain tested in the examples are also expected to be effective for treating or preventing cancer in the therapeutic combination of the invention. In certain embodiments, the bacterial strain for use in the therapeutic combination of the invention has a 16s rRNA sequence that is at least 95%, 96%, 97%, 98%, 99%, 99.5% or 99.9% identical to the 16s rRNA sequence of a bacterial strain of Enterococcus gallinarum. Preferably, the bacterial strain for use in the therapeutic combination of the invention has a 16s rRNA sequence that is at least 95%, 96%, 97%, 98%, 99%, 99.5% or 99.9% identical to SEQ ID NO:1 or 2. Preferably, the sequence identity is to SEQ ID NO:2. Preferably, the bacterial strain for use in the therapeutic combination of the invention has the 16s rRNA sequence represented by SEQ ID NO:2.

Bacterial strains that are biotypes of the bacterium deposited under accession number 42488 are also expected to be effective for treating or preventing cancer in the context of the therapeutic combination of the invention. A biotype is a closely related strain that has the same or very similar physiological and biochemical characteristics.

Strains that are biotypes of the bacterium deposited under accession number NCIMB 42488 and that are suitable for use in the therapeutic combination of the invention may be identified by sequencing other nucleotide sequences for the bacterium deposited under accession number NCIMB 42488. For example, substantially the whole genome may be sequenced and a biotype strain for use in the therapeutic combination of the invention may have at least 95%, 96%, 97%, 98%, 99%, 99.5% or 99.9% sequence identity across at least 80% of its whole genome (e.g. across at least 85%, 90%, 95% or 99%, or across its whole genome). For example, in some embodiments, a biotype strain has at least 98% sequence identity across at least 98% of its genome or at least 99% sequence identity across 99% of its genome. Other suitable sequences for use in identifying biotype strains may include hsp60 or repetitive sequences such as BOX, ERIC, (GTG)₅, or REP or [18]. Biotype strains may have sequences with at least 95%, 96%, 97%, 98%, 99%, 99.5% or 99.9% sequence identity to the corresponding sequence of the bacterium deposited under accession number NCIMB 42488. In some embodiments, a biotype strain has a sequence with at least 95%, 96%, 97%, 98%, 99%, 99.5% or 99.9% sequence identity to the corresponding sequence of strain MRX518 deposited as NCIMB 42488 and comprises a 16S rRNA sequence that is at least 99% identical (e.g. at least 99.5% or at least 99.9% identical) to SEQ ID NO:2. In some embodiments, a biotype strain has a sequence with at least 95%, 96%, 97%, 98%, 99%, 99.5% or 99.9% sequence identity to the corresponding sequence of strain MRX518 deposited as NCIMB 42488 and has the 16S rRNA sequence of SEQ ID NO:2.

In certain embodiments, the bacterial strain for use in the therapeutic combination of the invention has a chromosome with sequence identity to SEQ ID NO:3 of WO2017/085520. In preferred embodiments, the bacterial strain for use in the therapeutic combination of the invention has a chromosome with at least 90% sequence identity (e.g. at least 92%, 94%, 95%, 96%, 97%, 98%, 99% or 100% sequence identity) to SEQ ID NO:3 of WO2017/085520 across at least 60% (e.g. at least 65%, 70%, 75%, 80%, 85%, 95%, 96%, 97%, 98%, 99% or 100%) of SEQ ID NO:3 of WO2017/085520. For example, the bacterial strain for use in the therapeutic combination of the invention may have a chromosome with at least 90% sequence identity to SEQ ID NO:3 of WO2017/085520 across 70% of SEQ ID NO:3 of WO2017/085520, or at least 90% sequence identity to SEQ ID NO:3 of WO2017/085520 across 80% of SEQ ID NO:3 of WO2017/085520, or at least 90% sequence identity to SEQ ID NO:3 of WO2017/085520 across 90% of SEQ ID NO:3 of WO2017/085520, or at least 90% sequence identity to SEQ ID NO:3 of WO2017/085520 across 100% of SEQ ID NO:3 of WO2017/085520, or at least 95% sequence identity to SEQ ID NO:3 of WO2017/085520 across 70% of SEQ ID NO:3 of WO2017/085520, or at least 95% sequence identity to SEQ ID NO:3 of WO2017/085520 across 80% of SEQ ID NO:3 of WO2017/085520, or at least 95% sequence identity to SEQ ID NO:3 of WO2017/085520 across 90% of SEQ ID NO:3 of WO2017/085520, or at least 95% sequence identity to SEQ ID NO:3 of WO2017/085520 across 100% of SEQ ID NO:3 of WO2017/085520, or at least 98% sequence identity to SEQ ID NO:3 of WO2017/085520 across 70% of SEQ ID NO:3 of WO2017/085520, or at least 98% sequence identity to SEQ ID NO:3 of WO2017/085520 across 80% of SEQ ID NO:3 of WO2017/085520, or at least 98% sequence identity to SEQ ID NO:3 of WO2017/085520 across 90% of SEQ ID NO:3 of WO2017/085520, or at least 98% identity to SEQ ID NO:3 of WO2017/085520 across 95% of SEQ ID NO:3 of WO2017/085520, or at least 98% sequence identity to SEQ ID NO:3 of WO2017/085520 across 100% of SEQ ID NO:3 of WO2017/085520, or at least 99.5% sequence identity to SEQ ID NO:3 of WO2017/085520 across 90% of SEQ ID NO:3 of WO2017/085520, or at least 99.5% identity to SEQ ID NO:3 of WO2017/085520 across 95% of SEQ ID NO:3 of WO2017/085520, or at least 99.5% identity to SEQ ID NO:3 of WO2017/085520 across 98% of SEQ ID NO:3 of WO2017/085520, or at least 99.5% sequence identity to SEQ ID NO:3 of WO2017/085520 across 100% of SEQ ID NO:3 of WO2017/085520.

In certain embodiments, the bacterial strain for use in the therapeutic combination of the invention has a plasmid with sequence identity to SEQ ID NO:4 of WO2017/085520. In preferred embodiments, the bacterial strain for use in the therapeutic combination of the invention has a plasmid with at least 90% sequence identity (e.g. at least 92%, 94%, 95%, 96%, 97%, 98%, 99% or 100% sequence identity) to SEQ ID NO:4 of WO2017/085520 across at least 60% (e.g. at least 65%, 70%, 75%, 80%, 85%, 95%, 96%, 97%, 98%, 99% or 100%) of SEQ ID NO:4 of WO2017/085520. For example, the bacterial strain for use in the therapeutic combination of the invention may have a plasmid with at least 90% sequence identity to SEQ ID NO:4 of WO2017/085520 across 70% of SEQ ID NO:4 of WO2017/085520, or at least 90% sequence identity to SEQ ID NO:4 of WO2017/085520 across 80% of SEQ ID NO:4 of WO2017/085520, or at least 90% sequence identity to SEQ ID NO:4 of WO2017/085520 across 90% of SEQ ID NO:4 of WO2017/085520, or at least 90% sequence identity to SEQ ID NO:4 of WO2017/085520 across 100% of SEQ ID NO:4 of WO2017/085520, or at least 95% sequence identity to SEQ ID NO:4 of WO2017/085520 across 70% of SEQ ID NO:4 of WO2017/085520, or at least 95% sequence identity to SEQ ID NO:4 of WO2017/085520 across 80% of SEQ ID NO:4 of WO2017/085520, or at least 95% sequence identity to SEQ ID NO:4 of WO2017/085520 across 90% of SEQ ID NO:4 of WO2017/085520, or at least 95% sequence identity to SEQ ID NO:4 of WO2017/085520 across 100% of SEQ ID NO:4 of WO2017/085520, or at least 98% sequence identity to SEQ ID NO:4 of WO2017/085520 across 70% of SEQ ID NO:4 of WO2017/085520, or at least 98% sequence identity to SEQ ID NO:4 of WO2017/085520 across 80% of SEQ ID NO:4 of WO2017/085520, or at least 98% sequence identity to SEQ ID NO:4 of WO2017/085520 across 90% of SEQ ID NO:4 of WO2017/085520, or at least 98% sequence identity to SEQ ID NO:4 of WO2017/085520 across 100% of SEQ ID NO:4 of WO2017/085520.

In certain embodiments, the bacterial strain for use in the therapeutic combination of the invention has a chromosome with sequence identity to SEQ ID NO:3 of WO2017/085520 and a plasmid with sequence identity to SEQ ID NO:4 of WO2017/085520.

In certain embodiments, the bacterial strain for use in the therapeutic combination of the invention has a chromosome with sequence identity to SEQ ID NO:3 of WO2017/085520, for example as described above, and a 16S rRNA sequence with sequence identity to any of SEQ ID NO:1 or 2, for example as described above, preferably with a 16s rRNA sequence that is at least 99% identical to SEQ ID NO: 2, more preferably which comprises the 16S rRNA sequence of SEQ ID NO:2, and optionally comprises a plasmid with sequence identity to SEQ ID NO:4 of WO2017/085520, as described above.

In certain embodiments, the bacterial strain for use in the therapeutic combination of the invention has a chromosome with sequence identity to SEQ ID NO:3 of WO2017/085520, for example as described above, and optionally comprises a plasmid with sequence identity to SEQ ID NO:4 of WO2017/085520, as described above, and is effective for treating or preventing cancer.

In certain embodiments, the bacterial strain for use in the therapeutic combination of the invention has a chromosome with sequence identity to SEQ ID NO:3 of WO2017/085520, for example as described above, and a 16S rRNA sequence with sequence identity to any of SEQ ID NOs: 1 or 2, for example as described above, and optionally comprises a plasmid with sequence identity to SEQ ID NO:4 of WO2017/085520, as described above, and is effective for treating or preventing cancer.

In certain embodiments, the bacterial strain for use in the therapeutic combination of the invention has a 16s rRNA sequence that is at least 99%, 99.5% or 99.9% identical to the 16s rRNA sequence represented by SEQ ID NO: 2 (for example, which comprises the 16S rRNA sequence of SEQ ID NO:2) and a chromosome with at least 95% sequence identity to SEQ ID NO:3 of WO2017/085520 across at least 90% of SEQ ID NO:3 of WO2017/085520, and optionally comprises a plasmid with sequence identity to SEQ ID NO:4 of WO2017/085520, as described above, and which is effective for treating or preventing cancer.

In certain embodiments, the bacterial strain for use in the therapeutic combination of the invention has a 16s rRNA sequence that is at least 99%, 99.5% or 99.9% identical to the 16s rRNA sequence represented by SEQ ID NO: 2 (for example, which comprises the 16S rRNA sequence of SEQ ID NO:2) and a chromosome with at least 98% sequence identity (e.g. at least 99% or at least 99.5% sequence identity) to SEQ ID NO:3 of WO2017/085520 across at least 98% (e.g. across at least 99% or at least 99.5%) of SEQ ID NO:3 of WO2017/085520, and optionally comprises a plasmid with sequence identity to SEQ ID NO:4 of WO2017/085520, as described above, and which is effective for treating or preventing cancer.

In certain embodiments, the bacterial strain for use in the therapeutic combination of the invention is a Enterococcus gallinarum and has a 16s rRNA sequence that is at least 99%, 99.5% or 99.9% identical to the 16s rRNA sequence represented by SEQ ID NO: 2 (for example, which comprises the 16S rRNA sequence of SEQ ID NO:2) and a chromosome with at least 98% sequence identity (e.g. at least 99% or at least 99.5% sequence identity) to SEQ ID NO:3 of WO2017/085520 across at least 98% (e.g. across at least 99% or at least 99.5%) of SEQ ID NO:3 of WO2017/085520, and optionally comprises a plasmid with sequence identity to SEQ ID NO:4 of WO2017/085520, as described above, and which is effective for treating or preventing cancer.

Alternatively, strains that are biotypes of the bacterium deposited under accession number NCIMB 42488 and that are suitable for use in the therapeutic combination of the invention may be identified by using the accession number NCIMB 42488 deposit and restriction fragment analysis and/or PCR analysis, for example by using fluorescent amplified fragment length polymorphism (FAFLP) and repetitive DNA element (rep)-PCR fingerprinting, or protein profiling, or partial 16S or 23s rDNA sequencing. In preferred embodiments, such techniques may be used to identify other Enterococcus gallinarum strains.

In certain embodiments, strains that are biotypes of the bacterium deposited under accession number NCIMB 42488 and that are suitable for use in the therapeutic combination of the invention are strains that provide the same pattern as the bacterium deposited under accession number NCIMB 42488 when analysed by amplified ribosomal DNA restriction analysis (ARDRA), for example when using Sau3AI restriction enzyme (for exemplary methods and guidance see, for example, [19]). Alternatively, biotype strains are identified as strains that have the same carbohydrate fermentation patterns as the bacterium deposited under accession number NCIMB 42488. In some embodiments, the carbohydrate fermentation pattern is determined using the API 50 CHL panel (bioMérieux). In some embodiments, the bacterial strain used in the therapeutic combination of the invention is:
(i) positive for fermentation of at least one of (e.g. at least 2, 3, 4, 5, 6, 7, 8, 9, 10, 11, 12, 13, 14, 15, 16, 17 or all of): L-arabinose, D-ribose, D-xylose, D-galactose, D-glucose, D-fructose, D-mannose, N-acetylglucosamine, amygdalin, arbutin, salicin, D-cellobiose, D-maltose, sucrose, D-trehalose, gentiobiose, D-tagatose and potassium gluconate; and/or
(ii) intermediate for fermentation of at least one of (e.g. at least 2, 3, 4 or all of): D-mannitol, Methyl-αD-glycopyranoside, D-lactose, starch, and L-fucose;
   preferably as determined by API 50 CHL analysis (preferably using the API 50 CHL panel from bioMérieux).

Other Enterococcus gallinarum strains that are useful in the compositions and methods of the invention, such as biotypes of the bacterium deposited under accession number NCIMB 42488, may be identified using any appropriate method or strategy, including the assays described in the examples. For instance, strains for use in the therapeutic combination of the invention may be identified by culturing in anaerobic YCFA and/or administering the bacteria to the type II collagen-induced arthritis mouse model and then assessing cytokine levels. In particular, bacterial strains that have similar growth patterns, metabolic type and/or surface antigens to the bacterium deposited under accession number NCIMB 42488 may be useful in the therapeutic combination of the invention. A useful strain will have comparable immune modulatory activity to the NCIMB 42488 strain. In particular, a biotype strain will elicit comparable effects on the cancer disease models to the effects shown in the Examples, which may be identified by using the culturing and administration protocols described in the Examples. According to some embodiments, a biotype strain that may be used in the therapeutic combination of the invention is a strain which is able to elicit comparable effects on the cancer disease models shown in the Examples when administered in the therapeutic combination or method of the invention.

In some embodiments, the bacterial strain used in the therapeutic combination of the invention is:
(i) Positive for at least one of (e.g. at least 2, 3, 4, 5, 6, 7 or all of): mannose fermentation, glutamic acid decarboxylase, arginine arylamidase, phenylalanine arylamidase, pyroglutamic acid arylamidase, tyrosine arylamidase, histidine arylamidase and serine arylamidase; and/or
(ii) Intermediate for at least one of (e.g. at least 2 or all of): β-galactosidase-6-phosphate, β-glucosidase and N-acetyl-β-glucosaminidase; and/or
(iii) Negative for at least one of (e.g. at least 2, 3, 4, 5, 6 or all of): Raffinose fermentation, Proline arylamidase, Leucyl glycine arylamidase, Leucine arylamidase, Alanine arylamidase, Glycine arylamidase and Glutamyl glutamic acid arylamidase,

preferably as determined by an assay of carbohydrate, amino acid and nitrate metabolism, and optionally an assay of alkaline phosphatase activity, more preferably as determined by Rapid ID 32A analysis (preferably using the Rapid ID 32A system from bioMérieux).

In some embodiments, the bacterial strain used in the therapeutic combination of the invention is:
(i) Negative for at least one of (e.g. at least 2, 3, or all 4 of) glycine arylamidase, raffinose fermentation, proline arylamidase, and leucine arylamidase, for example, as determined by an assay of carbohydrate, amino acid and nitrate metabolism, preferably as determined by Rapid ID 32A analysis (preferably using the Rapid ID 32A system from bioMérieux); and/or
(ii) Intermediate positive for fermentation of L-fucose, preferably as determined by API 50 CHL analysis (preferably using the API 50 CHL panel from bioMérieux).

In some embodiments, the bacterial strain used in the therapeutic combination of the invention is an extracellular ATP producer, for example one which produces 6-6.7 ng/μl (for example, 6.1-6.6 ng/μl or 6.2-6.5 ng/μl or 6.33±0.10 ng/μl) of ATP as measured using the ATP Assay Kit (Sigma-Aldrich, MAK190). Bacterial extracellular ATP can have pleiotropic effects including activation of T cell-receptor mediated signalling (Schenk et al., 2011), promotion of intestinal Th17 cell differentiation (Atarashi et al., 2008) and induction of secretion of the pro-inflammatory mediator IL-1β by activating the NLRP3 inflammasome (Karmarkar et al., 2016). Accordingly, a bacterial strain which is an extracellular ATP producer is useful for treating or preventing cancer in the context of the therapeutic combination and method of the invention.

In some embodiments, the bacterial strain for use in the therapeutic combination of the invention comprises one or more of the following three genes: Mobile element protein; Xylose ABC transporter, permease component; and FIG00632333: hypothetical protein. For example, in certain embodiments, the bacterial strain for use in the therapeutic combination of the invention comprises genes encoding Mobile element protein and Xylose ABC transporter, permease component; Mobile element protein and FIG00632333: hypothetical protein; Xylose ABC transporter, permease component and FIG00632333: hypothetical protein; or Mobile element protein, Xylose ABC transporter, permease component, and FIG00632333: hypothetical protein.

A particularly preferred strain of the therapeutic combination of the invention is the Enterococcus gallinarum strain deposited under accession number NCIMB 42488. This is the exemplary MRX518 strain tested in the examples and shown to be effective for treating disease. The invention provides, according to some embodiments, a bacterial composition as part of the therapeutic combination of the invention, comprising a cell of the Enterococcus gallinarum strain deposited under accession number NCIMB 42488, or a derivative thereof. A derivative of the strain deposited under accession number NCIMB 42488 may be a daughter strain (progeny) or a strain cultured (subcloned) from the original.

A derivative of a strain of the composition comprised in the therapeutic combination of the invention may be modified, for example at the genetic level, without ablating the biological activity. In particular, a derivative strain of the therapeutic combination of the invention is therapeutically active. A derivative strain will have comparable immune modulatory activity to the original NCIMB 42488 strain. In particular, a derivative strain will elicit comparable effects on the cancer disease models when combined with pembrolizumab to the effects shown in the Examples, which may be identified by using the culturing and administration protocols described in the Examples. A derivative of the NCIMB 42488 strain will generally be a biotype of the NCIMB 42488 strain.

References to cells of the Enterococcus gallinarum strain deposited under accession number NCIMB 42488 encompass any cells that have the same safety and therapeutic efficacy characteristics as the strains deposited under accession number NCIMB 42488, and such cells are encompassed by the therapeutic combination of the invention. Thus, in some embodiments, reference to cells of the Enterococcus gallinarum strain deposited under accession number NCIMB 42488 refers only to the MRX518 strain deposited under NCIMB 42488 and does not refer to a bacterial strain that was not deposited under NCIMB 42488. In some embodiments, reference to cells of the Enterococcus gallinarum strain deposited under accession number NCIMB 42488 refers to cells that have the same safety and therapeutic efficacy characteristics as the strains deposited under accession number NCIMB 42488, but which are not the strain deposited under NCIMB 42488.

In preferred embodiments, the bacterial strains in the compositions of the invention are viable and capable of partially or totally colonising the intestine.

Treating Cancer

In preferred embodiments, the therapeutic combinations of the invention are for use in treating or preventing cancer. The examples demonstrate that administration of the therapeutic combinations of the invention can lead to a reduction in tumour growth.

In certain embodiments, treatment with the therapeutic combinations of the invention results in a reduction in tumour size or a reduction in tumour growth. In certain embodiments, the therapeutic combinations of the invention are for use in reducing tumour size or reducing tumour growth. The therapeutic combinations of the invention may be effective for reducing tumour size or growth. In certain embodiments, the therapeutic combinations of the invention are for use in patients with solid tumours. In certain embodiments, the therapeutic combinations of the invention are for use in reducing or preventing angiogenesis in the treatment of cancer. The therapeutic combinations of the invention may have an effect on the immune or inflammatory systems, which have central roles in angiogenesis. In certain embodiments, the therapeutic combinations of the invention are for use in preventing metastasis.

In certain embodiments, the therapeutic combinations of the invention are for use in treating or preventing breast cancer. The examples demonstrate that the therapeutic combinations of the invention may be effective for treating breast cancer. In certain embodiments, the therapeutic combinations of the invention are for use in reducing tumour size, reducing tumour growth, or reducing angiogenesis in the treatment of breast cancer. In preferred embodiments the cancer is mammary carcinoma. In preferred embodiments the cancer is stage IV breast cancer.

In certain embodiments, the therapeutic combinations of the invention are for use in treating or preventing lung cancer. The examples demonstrate that the therapeutic combinations of the invention may be effective for treating lung cancer. In certain embodiments, the therapeutic combinations of the invention are for use in reducing tumour size, reducing tumour growth, or reducing angiogenesis in the treatment of lung cancer. In preferred embodiments the cancer is lung carcinoma.

In certain embodiments, the therapeutic combinations of the invention are for use in treating or preventing liver cancer. The examples demonstrate that the therapeutic combinations of the invention may be effective for treating liver cancer. In certain embodiments, the therapeutic combinations of the invention are for use in reducing tumour size, reducing tumour growth, or reducing angiogenesis in the treatment of liver cancer. In preferred embodiments the cancer is hepatoma (hepatocellular carcinoma).

In certain embodiments, the therapeutic combinations of the invention are for use in treating or preventing colon cancer. The examples demonstrate that the therapeutic combinations of the invention have an effect on colon cancer cells and may be effective for treating colon cancer. In certain embodiments, the therapeutic combinations of the invention are for use in reducing tumour size, reducing tumour growth, or reducing angiogenesis in the treatment of colon cancer. In preferred embodiments the cancer is colorectal adenocarcinoma.

In certain embodiments, the therapeutic combinations of the invention are for use in treating or preventing kidney cancer (also referred to herein as renal cancer). The examples demonstrate that the therapeutic combinations of the invention have an effect on renal cancer cells and may be effective for treating renal cancer. In certain embodiments, the therapeutic combinations of the invention are for use in reducing tumour size, reducing tumour growth, or reducing angiogenesis in the treatment of renal cancer. In preferred embodiments the cancer is renal cell carcinoma or transitional cell carcinoma.

In certain embodiments, the therapeutic combinations of the invention are for use in treating or preventing melanoma. According to some embodiments, the therapeutic combinations of the invention have an effect on melanocytes and may be effective for treating melanoma. In certain embodiments, the therapeutic combinations of the invention are for use in reducing tumour size, reducing tumour growth, or reducing angiogenesis in the treatment of melanoma.

In some embodiments, the cancer is of the intestine. In some embodiments, the cancer is of a part of the body which is not the intestine. In some embodiments, the cancer is not cancer of the intestine. In some embodiments, the cancer is not colorectal cancer. In some embodiments, the cancer is not cancer of the small intestine. In some embodiments, the treating or preventing occurs at a site other than at the intestine. In some embodiments, the treating or preventing occurs at the intestine and also at a site other than at the intestine.

In certain embodiments, the therapeutic combinations of the invention are for use in treating or preventing carcinoma. The examples demonstrate that the therapeutic combinations of the invention may be effective for treating numerous types of carcinoma. In certain embodiments, the therapeutic combinations of the invention are for use in treating or preventing non-immunogenic cancer. The examples demonstrate that the therapeutic combinations of the invention may be effective for treating non-immunogenic cancers.

The therapeutic effects of the bacterial compositions of the invention on cancer, in the context of the therapeutic combinations of the invention, may be mediated by a pro-inflammatory mechanism. Examples 2, 4 and 5 demonstrate that the expression of a number of pro-inflammatory cytokines may be increased following administration of MRX518. Inflammation can have a cancer-suppressive effect [20] and pro-inflammatory cytokines such as TNFα are being investigated as cancer therapies [21]. The up-regulation of genes such as TNF shown in the examples may indicate that the bacterial compositions of the invention may be useful for treating cancer via a similar mechanism. The up-regulation of CXCR3 ligands (CXCL9, CXCL10) and IFNγ-inducible genes (IL-32) may indicate that the bacterial compositions of the invention elicit an IFNγ-type response. IFNγ is a potent macrophage-activating factor that can stimulate tumirocidal activity [22], and CXCL9 and CXCL10, for example, also have anti-cancer effects [23-25]. Therefore, in certain embodiments, the bacterial compositions of the invention, when used in the context of the therapeutic combination of the invention, are for use in promoting inflammation in the treatment of cancer. In preferred embodiments, the compositions of the invention, when used in the context of the therapeutic combination of the invention, are for use in promoting Th1 inflammation in the treatment of cancer. Th1 cells produce IFNγ and have potent anti-cancer effects [20]. In certain embodiments, the compositions of the invention, when used in the context of the therapeutic combination of the invention, are for use in treating an early-stage cancer, such as a cancer that has not metastasized, or a stage 0 or stage 1 cancer. Promoting inflammation may be more effective against early-stage cancers [°]. In certain embodiments, the compositions of the invention, when used in the context of the therapeutic combination of the invention, are for use in promoting inflammation to enhance the effect of pembrolizumab. In certain embodiments, the treatment or prevention of cancer comprises increasing the level of expression of one or more cytokines. For example, in certain embodiments, the treatment or prevention of cancer comprises increasing the level of expression of one or more of IL-1β, IL-6 and TNF-α, for example, IL-1β and IL-6, IL-1β and TNF-α, IL-6 and TNF-α or all three of IL-1β, IL-6 and TNF-α. Increases in levels of expression of any of IL-1β, IL-6 and TNF-α are known to be indicative of efficacy in treatment of cancer.

Examples 4 and 5 demonstrate that when a bacterial strain as described herein is used in combination with lipopolysaccharide (LPS), there is a synergistic increase in IL-1β. LPS is known to elicit a pro-inflammatory effect. Thus, in certain embodiments, the treatment or prevention of cancer comprises using a bacterial strain as described herein in combination with an agent that upregulates IL-1β. In certain embodiments, the treatment or prevention of cancer comprises using a bacterial strain as described herein in combination with LPS. Accordingly, the therapeutic combination of the invention may additionally comprise an agent that upregulates IL-1β. Accordingly, the bacterial composition of the invention may additionally comprise LPS.

In certain embodiments, the therapeutic combinations of the invention are for use in treating a patient that has previously received chemotherapy. In certain embodiments, the therapeutic combinations of the invention are for use in treating a patient that has not tolerated a chemotherapy treatment. The therapeutic combinations of the invention may be particularly suitable for such patients. In other embodiments, the therapeutic combinations of the invention are for use in treating a cancer patient who was non responsive to a prior treatment with an immune checkpoint inhibitor. In other embodiments, the therapeutic combinations of the invention are for use in treating a cancer patient who was non responsive to a prior treatment with a PD-1 inhibitor, such as, but not limited to, Pembrolizumab. Without wishing to be bound by theory or mechanism, it is believed that the bacterial composition of the invention is able to stimulate the subject's immune system through a different mechanism to that of pembrolizumab, thus providing a complementary mechanism to treat patients which are non-responsive to immune checkpoint inhibitors.

According to some embodiments, treatment of cancer using the therapeutic combination of the invention results is more effective than using pembrolizumab alone as measured by the RECIST (Response Evaluation Criteria In Solid Tumours) criteria or the irRECIST (immune-related Response Evaluation Criteria In Solid Tumours) criteria. According to some embodiments, treatment of cancer using the therapeutic combination of the invention results is more effective than using the bacterial composition alone as measured by the RECIST (Response Evaluation Criteria In Solid Tumours) criteria or the irRECIST (immune-related Response Evaluation Criteria In Solid Tumours) criteria. According to some embodiment, treatment of cancer using the therapeutic combination of the invention results in synergistic clinical effects as compared to treatment with pembrolizumab alone or the bacterial composition alone, as measured by the RECIST (Response Evaluation Criteria In Solid Tumours) criteria or the irRECIST (immune-related Response Evaluation Criteria In Solid Tumours) criteria.

In certain embodiments, the therapeutic combinations of the invention are for preventing relapse. The bacterial compositions, in the context of the therapeutic combinations of the invention, may be suitable for long-term administration. In certain embodiments, the therapeutic combinations of the invention are for use in preventing progression of cancer.

In certain embodiments, the therapeutic combinations of the invention are for use in treating non-small-cell lung carcinoma (NSCLC). In certain embodiments, the therapeutic combinations of the invention are for use in treating small-cell lung carcinoma. In certain embodiments, the therapeutic combinations of the invention are for use in treating squamous-cell carcinoma. In certain embodiments, the therapeutic combinations of the invention are for use in treating adenocarcinoma. In certain embodiments, the therapeutic combinations of the invention are for use in treating glandular tumors, carcinoid tumors, or undifferentiated carcinomas.

In certain embodiments, the therapeutic combinations of the invention are for use in treating hepatoblastoma, cholangiocarcinoma, cholangiocellular cystadenocarcinoma or liver cancer resulting from a viral infection.

In certain embodiments, the therapeutic combinations of the invention are for use in treating invasive ductal carcinoma, ductal carcinoma in situ or invasive lobular carcinoma.

In further embodiments, the therapeutic combinations of the invention are for use in treating or preventing acute lymphoblastic leukemia (ALL), acute myeloid leukemia, adrenocortical carcinoma, basal-cell carcinoma, bile duct cancer, bladder cancer, bone tumor, osteosarcoma/malignant fibrous histiocytoma, brainstem glioma, brain tumor, cerebellar astrocytoma, cerebral astrocytoma/malignant glioma, ependymoma, medulloblastoma, supratentorial primitive neuroectodermal tumors, breast cancer, bronchial adenomas/carcinoids, Burkitt's lymphoma, carcinoid tumor, cervical cancer, chronic lymphocytic leukemia, chronic myelogenous leukemia, chronic myeloproliferative disorders, colon cancer, cutaneous T-cell lymphoma, endometrial cancer, ependymoma, esophageal cancer, Ewing's sarcoma, intraocular melanoma, retinoblastoma, gallbladder cancer, gastric cancer, gastrointestinal carcinoid tumor, gastrointestinal stromal tumor (GIST), germ cell tumor, glioma, childhood visual pathway and hypothalamic, Hodgkin lymphoma, melanoma, islet cell carcinoma, Kaposi sarcoma, renal cell cancer, laryngeal cancer, leukaemias, lymphomas, mesothelioma, neuroblastoma, non-Hodgkin lymphoma, oropharyngeal cancer, osteosarcoma, ovarian cancer, pancreatic cancer, parathyroid cancer, pharyngeal cancer, pituitary adenoma, plasma cell neoplasia, prostate cancer, renal cell carcinoma, retinoblastoma, sarcoma, testicular cancer, thyroid cancer, or uterine cancer.

According to some embodiments, the therapeutic combinations are for use in treating or preventing cancer selected from the group consisting of: melanoma, NSCLC, bladder cancer and head-and-neck cancer.

In certain embodiments, the therapeutic combinations of the invention comprises an additional anticancer agent. According to some embodiments, the additional anticancer agent is selected from: a targeted antibody immunotherapy, a CAR-T cell therapy, an oncolytic virus, or a cytostatic drug.

Modes of Administration

Preferably, the bacterial compositions of the invention are to be administered to the gastrointestinal tract in order to enable delivery to and/or partial or total colonisation of the intestine with the bacterial strain of the invention. Generally, the bacterial compositions of the invention are administered orally, but they may be administered rectally, intranasally, or via buccal or sublingual routes.

In certain embodiments, the bacterial compositions of the invention may be administered as a foam, as a spray or a gel.

In certain embodiments, the bacterial compositions of the invention may be administered as a suppository, such as a rectal suppository, for example in the form of a Theobroma oil (cocoa butter), synthetic hard fat (e.g. suppocire, witepsol), glycero-gelatin, polyethylene glycol, or soap glycerin composition.

In certain embodiments, the bacterial composition of the invention is administered to the gastrointestinal tract via a tube, such as a nasogastric tube, orogastric tube, gastric tube, jejunostomy tube (J tube), percutaneous endoscopic gastrostomy (PEG), or a port, such as a chest wall port that provides access to the stomach, jejunum and other suitable access ports.

The bacterial compositions of the invention may be administered once, or they may be administered sequentially as part of a treatment regimen. In certain embodiments, the bacterial compositions of the invention are to be administered daily.

In certain embodiments of the invention, treatment according to the invention is accompanied by assessment of the patient's gut microbiota. Treatment may be repeated if delivery of and/or partial or total colonisation with the strain of the bacterial composition of the invention is not achieved such that efficacy is not observed, or treatment may be ceased if delivery and/or partial or total colonisation is successful and efficacy is observed. According to some embodiments, the bacterial composition of the invention is administered to the subject prior to first administration with the pembrolizumab of the therapeutic combination of the invention. According to some embodiments, the subject's gut microbiota is assessed after administration of the bacterial composition and before first administration of pembrolizumab, such that pembrolizumab is administered only after delivery and/or partial or total colonisation with the strain of the bacterial strain in the composition is achieved. In certain embodiments, the therapeutic combination of the invention may be administered to a pregnant animal, for example a mammal such as a human in order to reduce the likelihood of cancer developing in her child in utero and/or after it is born.

The therapeutic combination of the invention may be administered to a patient that has been diagnosed with cancer, or that has been identified as being at risk of a cancer. The therapeutic combination may also be administered as a prophylactic measure to prevent the development of cancer in a healthy patient.

The therapeutic combination of the invention may be administered to a patient that has been identified as having an abnormal gut microbiota. For example, the patient may have reduced or absent colonisation by Enterococcus gallinarum.

The bacterial compositions of the invention may be administered as a food product, such as a nutritional supplement.

Generally, the therapeutic combinations of the invention are for the treatment of humans, although they may be used to treat animals including monogastric mammals such as poultry, pigs, cats, dogs, horses or rabbits. The therapeutic combinations of the invention may be useful for enhancing the growth and performance of animals. If the bacterial composition is administered to animals, oral gavage may be used.

According to some embodiments, pembrolizumab is administered intravenously. According to some embodiments, pembrolizumab which is administered intravenously is in a composition which optionally further comprises at least one pharmaceutically compatible carrier or excipient. According to some embodiments, pembrolizumab is administered intravenously every about one, two, three or four weeks, preferably every three weeks.

According to some embodiments, the bacterial composition and the pembrolizumab of the therapeutic combination of the invention are administered using different administration routes. According to some embodiments, the bacterial composition is administered orally whereas the pembrolizumab of the therapeutic combination of the invention is administered using a different route. According to some embodiments, the pembrolizumab of the therapeutic combination is administered intravenously whereas the bacterial composition is administered orally.

According to some embodiments, the bacterial composition is administered to the subject prior to a first administration of pembrolizumab to the subject. According to some embodiments, the bacterial composition is administered to the subject prior to a first administration of pembrolizumab to the subject; wherein the bacterial composition is administered until delivery and/or partial or total colonisation with the strain of the bacterial strain in the composition is achieved. According to some embodiments, the bacterial composition is administered to the subject prior to a first administration of pembrolizumab to the subject; wherein the bacterial composition is administered until sufficient modulation of biomarkers occurs such that pembrolizumab is capable of treating a cancer patient who was previously non-responsive to ICI treatment. According to some embodiments, the bacterial composition is administered to the subject for at least one, two, three or four weeks prior to first administration of pembrolizumab. According to some embodiments, the bacterial composition is administered to the subject for about two weeks prior to first administration of pembrolizumab. According to some embodiments, the bacterial composition is administered to the subject for at least one, two, three or four weeks prior to first administration of pembrolizumab and is not administered to the subject in parallel to administration of pembrolizumab.

According to some embodiments, the first administration of the bacterial composition in the therapeutic combination of the invention is prior to the first administration of pembrolizumab. According to some embodiments, the first administration of pembrolizumab occurs no more than about 1, 2, 3, 4, 5, 6 or 7 days following administration of the bacterial composition.

According to some embodiments of the method and therapeutic combination of the invention, the bacterial composition is administered to the subject at least partially in parallel to administration of pembrolizumab to the subject. According to some embodiments, the bacterial composition is administered to the subject for a first time period, followed by administration of pembrolizumab to the subject for a second time period; wherein the bacterial composition is optionally further administered to the subject for at least part of said second time period, optionally all through the second time period. According to certain embodiments, the bacterial composition is administered to the subject for a first time period, such as, but not limited to, for about two weeks, followed by administration of pembrolizumab to the subject for a second time period, such as, but not limited to, for about three weeks. According to certain embodiments, the bacterial composition is administered to the subject for a first time period, such as, but not limited to, for about two weeks, followed by administration of pembrolizumab to the subject for a second time period, such as, but not limited to, for about three weeks; wherein the bacterial composition is further administered to the subject for at least part of said second time period, preferably all through the second time period.

According to some embodiments, the bacterial composition and pembrolizumab are not administered at the same frequency. In a non-limiting example, pembrolizumab is administered intravenously every three weeks, whereas the bacterial composition is administered orally every day or every other day. According to some embodiments, the bacterial composition is administered to the subject for a first time period, followed by administration of pembrolizumab to the subject for a second time period; wherein the bacterial composition is optionally further administered to the subject for at least part of said second time period; and wherein the frequency of administration of the bacterial composition is different in the first time period and second time period.

Bacterial Compositions of the Therapeutic Combination of the Invention

Generally, the composition comprised in the therapeutic combination of the invention comprises bacteria. In preferred embodiments of the invention, the bacterial composition is formulated in freeze-dried form. For example, the bacterial composition of the invention may comprise granules or gelatin capsules, for example hard gelatin capsules, comprising a bacterial strain of the invention.

Preferably, the bacterial composition of the invention comprises lyophilised bacteria. Lyophilisation of bacteria is a well-established procedure and relevant guidance is available in, for example, references [26-28].

Alternatively, the bacterial composition of the invention may comprise a live, active bacterial culture.

In some embodiments, the bacterial strain in the bacterial composition of the invention has not been inactivated, for example, has not been heat-inactivated. In some embodiments, the bacterial strain in the bacterial composition of the invention has not been killed, for example, has not been heat-killed. In some embodiments, the bacterial strain in the bacterial composition of the invention has not been attenuated, for example, has not been heat-attenuated. For example, in some embodiments, the bacterial strain in the bacterial composition of the invention has not been killed, inactivated and/or attenuated. For example, in some embodiments, the bacterial strain in the bacterial composition of the invention is live. For example, in some embodiments, the bacterial strain in the bacterial composition of the invention is viable. For example, in some embodiments, the bacterial strain in the bacterial composition of the invention is capable of partially or totally colonising the intestine. For example, in some embodiments, the bacterial strain in the bacterial composition of the invention is viable and capable of partially or totally colonising the intestine.

In some embodiments, the bacterial composition comprises a mixture of live bacterial strains and bacterial strains that have been killed.

In preferred embodiments, the bacterial composition of the therapeutic combination of the invention is encapsulated to enable delivery of the bacterial strain to the intestine. Encapsulation protects the bacterial composition from degradation until delivery at the target location through, for example, rupturing with chemical or physical stimuli such as pressure, enzymatic activity, or physical disintegration, which may be triggered by changes in pH. Any appropriate encapsulation method may be used. Exemplary encapsulation techniques include entrapment within a porous matrix, attachment or adsorption on solid carrier surfaces, self-aggregation by flocculation or with cross-linking agents, and mechanical containment behind a microporous membrane or a microcapsule. Guidance on encapsulation that may be useful for preparing compositions of the invention is available in, for example, references [29] and [30].

The bacterial composition may be administered orally and may be in the form of a tablet, capsule or powder. Encapsulated products are preferred because Enterococcus gallinarum are anaerobes. Other ingredients (such as vitamin C, for example), may be included as oxygen scavengers and prebiotic substrates to improve the delivery and/or partial or total colonisation and survival in vivo. Alternatively, the probiotic composition of the invention may be administered orally as a food or nutritional product, such as milk or whey based fermented dairy product, or as a pharmaceutical product.

The bacterial composition may be formulated as a probiotic.

A bacterial composition of the invention includes a therapeutically effective amount of a bacterial strain of the invention. A therapeutically effective amount of a bacterial strain is sufficient to exert a beneficial effect upon a patient. A therapeutically effective amount of a bacterial strain may be sufficient to result in delivery to and/or partial or total colonisation of the patient's intestine.

A suitable daily dose of the bacteria, for example for an adult human, may be from about 1×10³ to about 1×10¹¹ colony forming units (CFU); for example, from about 1×10⁷ to about 1×10¹⁰ CFU; in another example from about 1×10⁶ to about 1×10¹⁰ CFU.

In certain embodiments, the bacterial composition contains the bacterial strain in an amount of from about 1×10⁶ to about 1×10¹¹ CFU/g, respect to the weight of the composition; for example, from about 1×10⁸ to about 1×10¹⁰ CFU/g. The dose may be, for example, 1 g, 3 g, 5 g, and 10 g.

A probiotic, such as the bacterial composition of the invention, may optionally be combined with at least one suitable prebiotic compound. A prebiotic compound is usually a non-digestible carbohydrate such as an oligo- or polysaccharide, or a sugar alcohol, which is not degraded or absorbed in the upper digestive tract. Known prebiotics include commercial products such as inulin and transgalacto-oligosaccharides.

In certain embodiments, the probiotic bacterial composition of the present invention includes a prebiotic compound in an amount of from about 1 to about 30% by weight, respect to the total weight composition, (e.g. from 5 to 20% by weight). Carbohydrates may be selected from the group consisting of: fructo-oligosaccharides (or FOS), short-chain fructo-oligosaccharides, inulin, isomalt-oligosaccharides, pectins, xylo-oligosaccharides (or XOS), chitosan-oligosaccharides (or COS), beta-glucans, arable gum modified and resistant starches, polydextrose, D-tagatose, acacia fibers, carob, oats, and citrus fibers. In one aspect, the prebiotics are the short-chain fructo-oligosaccharides (for simplicity shown herein below as FOSs-c.c); said FOSs-c.c. are not digestible carbohydrates, generally obtained by the conversion of the beet sugar and including a saccharose molecule to which three glucose molecules are bonded.

The bacterial compositions of the invention may comprise pharmaceutically acceptable excipients or carriers. Examples of such suitable excipients may be found in the reference [31]. Acceptable carriers or diluents for therapeutic use are well known in the pharmaceutical art and are described, for example, in reference [32]. Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may be also used.

The bacterial compositions of the invention may be formulated as a food product. For example, a food product may provide nutritional benefit in addition to the therapeutic effect of the invention, such as in a nutritional supplement. Similarly, a food product may be formulated to enhance the taste of the composition of the invention or to make the composition more attractive to consume by being more similar to a common food item, rather than to a pharmaceutical composition. In certain embodiments, the composition of the invention is formulated as a milk-based product. The term “milk-based product” means any liquid or semi-solid milk- or whey-based product having a varying fat content. The milk-based product can be, e.g., cow's milk, goat's milk, sheep's milk, skimmed milk, whole milk, milk recombined from powdered milk and whey without any processing, or a processed product, such as yoghurt, curdled milk, curd, sour milk, sour whole milk, butter milk and other sour milk products. Another important group includes milk beverages, such as whey beverages, fermented milks, condensed milks, infant or baby milks; flavoured milks, ice cream; milk-containing food such as sweets.

In certain embodiments, the bacterial compositions of the invention contain a single bacterial strain or species and do not contain any other bacterial strains or species. Such bacterial compositions may comprise only de minimis or biologically irrelevant amounts of other bacterial strains or species. Such bacterial compositions may be a culture that is substantially free from other species of organism. Thus, in some embodiments, the bacterial composition of the therapeutic combination comprises one or more strains from the species Enterococcus gallinarum, and does not contain bacteria from any other species or comprises only de minimis or biologically irrelevant amounts of bacteria from another species.

In some embodiments, the bacterial compositions of the invention comprise more than one bacterial strain or species. For example, in some embodiments, the bacterial compositions of the invention comprise more than one strain from within the same species (e.g. more than 1, 2, 3, 4, 5, 6, 7, 8, 9, 10, 15, 20, 25, 30, 35, 40 or 45 strains), and, optionally, do not contain bacteria from any other species. In some embodiments, the bacterial compositions of the invention comprise less than 50 strains from within the same species (e.g. less than 45, 40, 35, 30, 25, 20, 15, 12, 10, 9, 8, 7, 6, 5, 4 or 3 strains), and, optionally, do not contain bacteria from any other species. In some embodiments, the bacterial compositions of the invention comprise 1-40, 1-30, 1-20, 1-19, 1-18, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 strains from within the same species and, optionally, do not contain bacteria from any other species. In some embodiments, the bacterial compositions of the invention comprise more than one species from within the same genus (e.g. more than 1, 2, 3, 4, 5, 6, 7, 8, 9, 10, 12, 15, 17, 20, 23, 25, 30, 35 or 40 species), and, optionally, do not contain bacteria from any other genus. In some embodiments, the bacterial compositions of the invention comprise less than 50 species from within the same genus (e.g. less than 50, 45, 40, 35, 30, 25, 20, 15, 12, 10, 8, 7, 6, 5, 4 or 3 species), and, optionally, do not contain bacteria from any other genus. In some embodiments, the bacterial compositions of the invention comprise 1-50, 1-40, 1-30, 1-20, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 species from within the same genus and, optionally, do not contain bacteria from any other genus. The bacterial composition for use in the combination of the invention may comprise any combination of the foregoing.

In some embodiments, the bacterial composition comprises a microbial consortium. For example, in some embodiments, the bacterial composition comprises the bacterial strain having a 16s rRNA sequence that is at least 95% identical to SEQ ID NO:2, for example, which is an Enterococcus gallinarum, as part of a microbial consortium. For example, in some embodiments, the bacterial strain is present in the bacterial composition in combination with one or more (e.g. at least 2, 3, 4, 5, 10, 15 or 20) other bacterial strains from other genera with which it can live symbiotically in vivo in the intestine. For example, in some embodiments, the bacterial composition comprises a bacterial strain having a 16s rRNA sequence that is at least 95% identical to SEQ ID NO:2, for example, which is an Enterococcus gallinarum, in combination with a bacterial strain from a different genus. In some embodiments, the microbial consortium comprises two or more bacterial strains obtained from a faeces sample of a single organism, e.g. a human. In some embodiments, the microbial consortium is not found together in nature. For example, in some embodiments, the microbial consortium comprises bacterial strains obtained from faeces samples of at least two different organisms. In some embodiments, the two different organisms are from the same species, e.g. two different humans, e.g. two different human infants. In some embodiments, the two different organisms are an infant human and an adult human. In some embodiments, the two different organisms are a human and a non-human mammal.

In some embodiments, the bacterial composition of the invention additionally comprises a bacterial strain that has the same safety and therapeutic efficacy characteristics as strain MRX518, but which is not MRX518 deposited as NCIMB 42488, or which is not an Enterococcus gallinarum.

In some embodiments, the bacterial strain for use in the bacterial composition is obtained from human infant faeces. In some embodiments in which the bacterial composition comprises more than one bacterial strain, all of the bacterial strains are obtained from human infant faeces or if other bacterial strains are present they are present only in de minimis amounts. The bacteria may have been cultured subsequent to being obtained from the human infant faeces and being used in the bacterial composition.

As mentioned above, in some embodiments, the one or more bacterial strains having a 16s rRNA sequence that is at least 95% identical to SEQ ID NO:2, for example which is an Enterococcus gallinarum, is/are the only therapeutically active agent(s) in the bacterial composition of the invention. In some embodiments, the bacterial strain(s) in the bacterial composition is/are the only therapeutically active agent(s) in the composition.

The bacterial compositions for use in accordance with the invention may or may not require marketing approval.

In certain embodiments, the invention provides the above bacterial composition, wherein said bacterial strain is lyophilised. In certain embodiments, the invention provides the above bacterial composition, wherein said bacterial strain is spray dried. In certain embodiments, the invention provides the above bacterial composition, wherein the bacterial strain is lyophilised or spray dried and wherein it is alive. In certain embodiments, the invention provides the above bacterial composition, wherein the bacterial strain is lyophilised or spray dried and wherein it is viable. In certain embodiments, the invention provides the above bacterial composition, wherein the bacterial strain is lyophilised or spray dried and wherein it is capable of partially or totally colonising the intestine. In certain embodiments, the invention provides the above bacterial composition, wherein the bacterial strain is lyophilised or spray dried and wherein it is viable and capable of partially or totally colonising the intestine.

In some cases, the lyophilised or spray dried bacterial strain is reconstituted prior to administration. In some cases, the reconstitution is by use of a diluent described herein.

The bacterial compositions of the invention can comprise pharmaceutically acceptable excipients, diluents or carriers.

In certain embodiments, the bacterial composition is a pharmaceutical composition comprising: a bacterial strain as used in the invention; and a pharmaceutically acceptable excipient, carrier or diluent; wherein the bacterial strain is in an amount sufficient to treat a disorder when administered to a subject in combination with pembrolizumab; and wherein the disorder is breast cancer. In preferred embodiments the cancer is mammary carcinoma. In preferred embodiments the cancer is stage IV breast cancer.

In certain embodiments, the bacterial composition is a pharmaceutical composition comprising: a bacterial strain as used in the invention; and a pharmaceutically acceptable excipient, carrier or diluent; wherein the bacterial strain is in an amount sufficient to treat a disorder when administered to a subject in combination with pembrolizumab; and wherein the disorder is lung cancer. In preferred embodiments the cancer is lung carcinoma.

In certain embodiments, the bacterial composition is a pharmaceutical composition comprising: a bacterial strain as used in the invention; and a pharmaceutically acceptable excipient, carrier or diluent; wherein the bacterial strain is in an amount sufficient to treat a disorder when administered to a subject in combination with pembrolizumab; and wherein the disorder is liver cancer. In preferred embodiments the cancer is hepatoma (hepatocellular carcinoma).

In certain embodiments, the bacterial composition is a pharmaceutical composition comprising: a bacterial strain of the invention; and a pharmaceutically acceptable excipient, carrier or diluent; wherein the bacterial strain is in an amount sufficient to treat a disorder when administered to a subject in combination with pembrolizumab; and wherein the disorder is colon cancer. In preferred embodiments the cancer is colorectal adenocarcinoma.

In certain embodiments, the bacterial composition is a pharmaceutical composition comprising: a bacterial strain of the invention; and a pharmaceutically acceptable excipient, carrier or diluent; wherein the bacterial strain is in an amount sufficient to treat a disorder when administered to a subject in combination with pembrolizumab; and wherein the disorder is carcinoma.

In certain embodiments, the bacterial composition is a pharmaceutical composition comprising: a bacterial strain of the invention; and a pharmaceutically acceptable excipient, carrier or diluent; wherein the bacterial strain is in an amount sufficient to treat a disorder when administered to a subject in combination with pembrolizumab; and wherein the disorder is a non-immunogenic cancer.

In certain embodiments, the bacterial composition is a pharmaceutical composition comprising: a bacterial strain of the invention; and a pharmaceutically acceptable excipient, carrier or diluent; wherein the bacterial strain is in an amount sufficient to treat a disorder when administered to a subject in combination with pembrolizumab; and wherein the disorder is selected from the group consisting of non-small-cell lung carcinoma, small-cell lung carcinoma, squamous-cell carcinoma, adenocarcinoma, glandular tumors, carcinoid tumors undifferentiated carcinomas.

In certain embodiments, the bacterial composition is a pharmaceutical composition comprising: a bacterial strain of the invention; and a pharmaceutically acceptable excipient, carrier or diluent; wherein the bacterial strain is in an amount sufficient to treat a disorder when administered to a subject in combination with pembrolizumab; and wherein the disorder is selected from the group consisting of hepatoblastoma, cholangiocarcinoma, cholangiocellular cystadenocarcinoma or liver cancer resulting from a viral infection.

In certain embodiments, the bacterial composition is a pharmaceutical composition comprising: a bacterial strain of the invention; and a pharmaceutically acceptable excipient, carrier or diluent; wherein the bacterial strain is in an amount sufficient to treat a disorder when administered to a subject in combination with pembrolizumab; and wherein the disorder is selected from the group consisting of invasive ductal carcinoma, ductal carcinoma in situ or invasive lobular carcinoma.

In certain embodiments, the bacterial composition is a pharmaceutical composition comprising: a bacterial strain of the invention; and a pharmaceutically acceptable excipient, carrier or diluent; wherein the bacterial strain is in an amount sufficient to treat a disorder when administered to a subject in combination with pembrolizumab; and wherein the disorder is selected from the group consisting of acute lymphoblastic leukemia (ALL), acute myeloid leukemia, adrenocortical carcinoma, basal-cell carcinoma, bile duct cancer, bladder cancer, bone tumor, osteosarcoma/malignant fibrous histiocytoma, brainstem glioma, brain tumor, cerebellar astrocytoma, cerebral astrocytoma/malignant glioma, ependymoma, medulloblastoma, supratentorial primitive neuroectodermal tumors, breast cancer, bronchial adenomas/carcinoids, Burkitt's lymphoma, carcinoid tumor, cervical cancer, chronic lymphocytic leukemia, chronic myelogenous leukemia, chronic myeloproliferative disorders, colon cancer, cutaneous T-cell lymphoma, endometrial cancer, ependymoma, esophageal cancer, Ewing's sarcoma, intraocular melanoma, retinoblastoma, gallbladder cancer, gastric cancer, gastrointestinal carcinoid tumor, gastrointestinal stromal tumor (GIST), germ cell tumor, glioma, childhood visual pathway and hypothalamic, Hodgkin lymphoma, melanoma, islet cell carcinoma, Kaposi sarcoma, renal cell cancer, laryngeal cancer, leukaemias, lymphomas, mesothelioma, neuroblastoma, non-Hodgkin lymphoma, oropharyngeal cancer, osteosarcoma, ovarian cancer, pancreatic cancer, parathyroid cancer, pharyngeal cancer, pituitary adenoma, plasma cell neoplasia, prostate cancer, renal cell carcinoma, retinoblastoma, sarcoma, testicular cancer, thyroid cancer, or uterine cancer.

In certain embodiments, the amount of the bacterial strain in the bacterial composition is from about 1×10³ to about 1×10¹¹ colony forming units per gram with respect to a weight of the composition.

In certain embodiments, the bacterial composition is administered at a dose of 1 g, 3 g, 5 g or 10 g.

In certain embodiments, the bacterial composition is administered by a method selected from the group consisting of oral, rectal, subcutaneous, nasal, buccal, and sublingual.

In certain embodiments, the bacterial composition comprises a carrier selected from the group consisting of lactose, starch, glucose, methyl cellulose, magnesium stearate, mannitol and sorbitol.

In certain embodiments, the invention provides the bacterial composition comprises a diluent selected from the group consisting of ethanol, glycerol and water.

In certain embodiments, the bacterial composition comprises an excipient selected from the group consisting of starch, gelatin, glucose, anhydrous lactose, free-flow lactose, beta-lactose, corn sweetener, acacia, tragacanth, sodium alginate, carboxymethyl cellulose, polyethylene glycol, sodium oleate, sodium stearate, magnesium stearate, sodium benzoate, sodium acetate and sodium chloride.

In certain embodiments, the bacterial composition further comprises at least one of a preservative, an antioxidant and a stabilizer.

In certain embodiments, the bacterial composition comprises a preservative selected from the group consisting of sodium benzoate, sorbic acid and esters of p-hydroxybenzoic acid.

In certain embodiments, when the bacterial composition is stored in a sealed container at about 4.0 or about 25.0 and the container is placed in an atmosphere having 50% relative humidity, at least 80% of the bacterial strain as measured in colony forming units, remains after a period of at least about: 1 month, 3 months, 6 months, 1 year, 1.5 years, 2 years, 2.5 years or 3 years.

In some embodiments, the bacterial composition of the invention is provided in a sealed container. In some embodiments, the sealed container is a sachet or bottle. In some embodiments, the bacterial composition of the invention is provided in a syringe.

The bacteria; composition may, in some embodiments, be provided as a pharmaceutical formulation. For example, the bacterial composition may be provided as a tablet or capsule. In some embodiments, the capsule is a gelatine capsule (“gel-cap”).

In some embodiments, the bacterial compositions of the invention are administered orally. In some embodiments, the bacterial compositions of the inventions are formulated in a pharmaceutical formulation suitable for oral administration. Oral administration may involve swallowing, so that the compound enters the gastrointestinal tract, and/or buccal, lingual, or sublingual administration by which the compound enters the blood stream directly from the mouth.

Pharmaceutical formulations suitable for oral administration include solid plugs, solid microparticulates, semi-solid and liquid (including multiple phases or dispersed systems) such as tablets; soft or hard capsules containing multi- or nano-particulates, liquids (e.g. aqueous solutions), emulsions or powders; lozenges (including liquid-filled); chews; gels; fast dispersing dosage forms; films; ovules; sprays; and buccal/mucoadhesive patches.

In some embodiments the pharmaceutical formulation is an enteric formulation, i.e. a gastro-resistant formulation (for example, resistant to gastric pH) that is suitable for delivery of the composition of the invention to the intestine by oral administration. Enteric formulations may be particularly useful when the bacteria or another component of the composition is acid-sensitive, e.g. prone to degradation under gastric conditions.

In some embodiments, the enteric formulation comprises an enteric coating. In some embodiments, the formulation is an enteric-coated dosage form. For example, the formulation may be an enteric-coated tablet or an enteric-coated capsule, or the like. The enteric coating may be a conventional enteric coating, for example, a conventional coating for a tablet, capsule, or the like for oral delivery. The formulation may comprise a film coating, for example, a thin film layer of an enteric polymer, e.g. an acid-insoluble polymer.

In some embodiments, the enteric formulation is intrinsically enteric, for example, gastro-resistant without the need for an enteric coating. Thus, in some embodiments, the formulation is an enteric formulation that does not comprise an enteric coating. In some embodiments, the formulation is a capsule made from a thermogelling material. In some embodiments, the thermogelling material is a cellulosic material, such as methylcellulose, hydroxymethylcellulose or hydroxypropylmethylcellulose (HPMC). In some embodiments, the capsule comprises a shell that does not contain any film forming polymer. In some embodiments, the capsule comprises a shell and the shell comprises hydroxypropylmethylcellulose and does not comprise any film forming polymer (e.g. see [33]). In some embodiments, the formulation is an intrinsically enteric capsule (for example, Vcaps® from Capsugel).

In some embodiments, the formulation is a soft capsule. Soft capsules are capsules which may, owing to additions of softeners, such as, for example, glycerol, sorbitol, maltitol and polyethylene glycols, present in the capsule shell, have a certain elasticity and softness. Soft capsules can be produced, for example, on the basis of gelatine or starch. Gelatine-based soft capsules are commercially available from various suppliers. Depending on the method of administration, such as, for example, orally or rectally, soft capsules can have various shapes, they can be, for example, round, oval, oblong or torpedo-shaped. Soft capsules can be produced by conventional processes, such as, for example, by the Scherer process, the Accogel process or the droplet or blowing process.

Culturing Methods

The bacterial strains for use in the present invention can be cultured using standard microbiology techniques as detailed in, for example, references [34-36].

The solid or liquid medium used for culture may be YCFA agar or YCFA medium. YCFA medium may include (per 100 ml, approximate values): Casitone (1.0 g), yeast extract (0.25 g), NaHCO₃ (0.4 g), cysteine (0.1 g), K₂HPO₄ (0.045 g), KH₂PO₄ (0.045 g), NaCl (0.09 g), (NH₄)₂SO₄ (0.09 g), MgSO₄.7H₂O (0.009 g), CaCl₂) (0.009 g), resazurin (0.1 mg), hemin (1 mg), biotin (1 μg), cobalamin (1 μg), p-aminobenzoic acid (3 μg), folic acid (5 μg), and pyridoxamine (15 μg).

Bacterial Strains for Use in Vaccine Compositions

The inventors have identified that the bacterial strains of the bacterial composition of the invention are useful for treating or preventing cancer when administered in combination with pembrolizumab. This is likely to be a result of the effect that the bacterial strains of the invention have on the host immune system. In certain embodiments, the bacterial strains are viable. In certain embodiments, the bacterial strains are capable of partially or totally colonising the intestine. In certain embodiments, the bacterial strains are viable and capable of partially or totally colonising the intestine. In other certain embodiments, the bacterial strains may be killed, inactivated or attenuated. In certain embodiments, the bacterial compositions are for administration via injection, such as via subcutaneous injection.

Pembrolizumab

The therapeutic combination of the invention comprises pembrolizumab. As described above, pembrolizumab is an antibody that inhibits immune checkpoints, thus enabling the body's immune system to attack cells that are recognized as the body's own cells, including cancer cells.

Pembrolizumab inhibits the interaction between the transmembrane receptor programmed cell death 1 protein (referred to as PDCD1, PD-1, PD1, or CD279) and its ligand, PD-1 ligand 1 (referred to as PD-L1, PDL1 or CD274) by binding to and blocking the PD-1 receptor, and is thus referred to as a PD-1 inhibitor.

Pembrolizumab is marketed by Merck under the commercial name KEYTRUDA®.

The terms “specific binding” or “specifically bind” as used herein refers to a non-random association between two molecules, i.e., antibody and antigen. According to some embodiments, pembrolizumab, via its antigen-binding domain, specifically binds to the antigen with a binding affinity (Kd) of <10″⁵M. Alternatively, pembrolizumab, via its antigen-binding domain, may bind to the antigen with a Kd of <10″⁶M or <10″ M. Kd, as used herein, refers to the ratio of the dissociation rate to the association rate (k_{off}/kₒₙ), and may be determined using any suitable methods known in the art.

According to some embodiments, the pembrolizumab of the therapeutic combination is administered systemically. According to some embodiments, the pembrolizumab is formulated for systemic administration.

According to another embodiment, administration systemically is through a parenteral route. According to some embodiments, preparations of the pembrolizumab of the invention for parenteral administration include sterile aqueous or non-aqueous solutions, suspensions, or emulsions, each representing a separate embodiment of the present invention.

According to some embodiments, parenteral administration is administration intravenously, intra-arterially, administering into a blood-vessel wall, intramuscularly, intraperitoneally, intradermally, intravitreally, transdermally or subcutaneously. Each of the abovementioned administration routes represents a separate embodiment of the present invention. According to some embodiments, the pembrolizumab of the therapeutic combination is administered intravenously.

According to some embodiments, systemic administration of pembrolizumab is through injection. For administration through injection, pembrolizumab may be formulated in an aqueous solution, for example in a physiologically compatible buffer, including, but not limited to, Hank's solution, Ringer's solution, or physiological salt buffer. Formulations for injection may be presented in unit dosage forms, for example, in ampoules, or in multi-dose containers with, optionally, an added preservative. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

According to some embodiment, parenteral administration is performed by bolus injection. According to other embodiments, parenteral administration is performed by continuous infusion. According to some embodiments, pembrolizumab is delivered in a controlled release system and is formulated for intravenous infusion, implantable osmotic pump, transdermal patch, liposomes, or other modes of administration. In one embodiment, a pump is used. In yet another embodiment, a controlled release system can be placed in proximity to the therapeutic target, thus requiring only a fraction of the systemic dose.

The Therapeutic Combination

According to some embodiments, provided herein is the therapeutic combination of the invention for use in a method of treating or preventing cancer in a subject. According to some embodiments, provided herein is the therapeutic combination of the invention for use in a method of treating cancer in a subject.

According to some embodiments, cancer to be treated or prevented using the therapeutic combination of the invention is selected from the group consisting of: melanoma, non-small cell lung carcinoma, bladder cancer and head-and-neck cancer. According to some embodiments, cancer to be treated or prevented using the therapeutic combination of the invention is selected from the group consisting of: breast cancer, lung cancer, colon cancer and liver cancer.

According to some embodiments, treating cancer relates to at least one of reducing tumour size or preventing tumour growth in a subject. According to some embodiments, the therapeutic combination or the method of the invention is for use in at least one of: reducing tumour size, reducing tumour growth, preventing metastasis or preventing angiogenesis in a subject afflicted with cancer.

According to some embodiments, the therapeutic combination of the invention comprises: (a) a composition comprising a bacterial strain of the species Enterococcus gallinarum; and (b) pembrolizumab.

According to some embodiments, the bacterial composition of the therapeutic combination does not contain bacteria from any other species other than Enterococcus gallinarum, or comprises only de minimis or biologically irrelevant amounts of bacteria from another species. According to some embodiments, the bacterial composition of the therapeutic combination contains only a single strain of the species Enterococcus gallinarum, and does not contain bacteria from any other species or comprises only de minimis or biologically irrelevant amounts of bacteria from another species. According to some embodiments, the bacterial composition of the therapeutic combination comprises the Enterococcus gallinarum strain deposited under accession number NCIMB 42488. According to some embodiments, the bacterial composition of the therapeutic combination comprises a single strain of the Enterococcus gallinarum species, deposited under accession number NCIMB 42488, and does not contain bacteria from any other species or comprises only de minimis or biologically irrelevant amounts of bacteria from another species.

According to some embodiments, pembrolizumab is in a composition, possibly comprising at least one pharmaceutically acceptable carrier and/or excipient.

According to some embodiments, the therapeutic combination of the invention comprises: (a) a composition comprising a bacterial strain of the species Enterococcus gallinarum, wherein the composition comprises a single strain of the Enterococcus gallinarum species, deposited under accession number NCIMB 42488, optionally wherein the composition does not contain bacteria from any other species or comprises only de minimis or biologically irrelevant amounts of bacteria from another species; and (b) pembrolizumab.

Preferably, the therapeutic combination of the invention comprises: (a) a composition comprising the bacterial strain of the species Enterococcus gallinarum, deposited under accession number NCIMB 42488; and (b) Pembrolizumab. According to some embodiments, provided herein is a therapeutic combination for use in a method of treating or preventing cancer in a subject, wherein the therapeutic combination comprises: (a) a composition comprising the bacterial strain of the species Enterococcus gallinarum, deposited under accession number NCIMB 42488; and (b) Pembrolizumab.

According to some embodiments, the therapeutic combination of the invention comprises: (a) a composition comprising a bacterial strain of the species Enterococcus gallinarum; and (b) pembrolizumab.

According to some embodiments, the therapeutic combination of the invention comprises: (a) a composition comprising a bacterial strain of the species Enterococcus gallinarum, optionally the strain deposited under accession number NCIMB 42488, optionally wherein the composition does not contain bacteria from any other species and/or strains or comprises only de minimis or biologically irrelevant amounts of bacteria from another species and/or strain; and (b) pembrolizumab.

According to some embodiments, provided herein is a method for treating and/or preventing cancer in a subject using any one of the therapeutic combinations disclosed herein. According to some embodiments, the present invention provides any one of the therapeutic combinations disclosed herein for use in treating and/or preventing cancer in a subject.

General

The practice of the present invention will employ, unless otherwise indicated, conventional methods of chemistry, biochemistry, molecular biology, immunology and pharmacology, within the skill of the art. Such techniques are explained fully in the literature. See, e.g., references [37] and [38-44], etc.

The term “comprising” encompasses “including” as well as “consisting” e.g. a composition “comprising” X may consist exclusively of X or may include something additional e.g. X+Y.

The term “about” in relation to a numerical value x is optional and means, for example, x+10%.

The word “substantially” does not exclude “completely” e.g. a composition which is “substantially free” from Y may be completely free from Y. Where necessary, the word “substantially” may be omitted from the definition of the invention.

References to a percentage sequence identity between two nucleotide sequences means that, when aligned, that percentage of nucleotides are the same in comparing the two sequences. This alignment and the percent homology or sequence identity can be determined using software programs known in the art, for example those described in section 7.7.18 of ref. [45]. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref [46].

Unless specifically stated, a process or method comprising numerous steps may comprise additional steps at the beginning or end of the method, or may comprise additional intervening steps. Also, steps may be combined, omitted or performed in an alternative order, if appropriate.

Various embodiments of the invention are described herein. It will be appreciated that the features specified in each embodiment may be combined with other specified features, to provide further embodiments. In particular, embodiments highlighted herein as being suitable, typical or preferred may be combined with each other (except when they are mutually exclusive).

### MODES FOR CARRYING OUT THE INVENTION

### Example 1—Efficacy of Bacterial Inocula in Mouse Models of Cancer

Summary

This study tested the efficacy of compositions comprising bacterial strains according to the invention in four tumor models.

Materials

Test substance—Bacterial strain #MRX518.

Reference substance—Anti-CTLA-4 antibody (clone: 9H10, catalog: BE0131, isotype: Syrian Hamster IgG1, Bioxcell).

Test and reference substances vehicles—Bacterial culture medium (Yeast extract, Casitone, Fatty Acid medium (YCFA)). Each day of injection to mice, antibody was diluted with PBS (ref: BE14-516F, Lonza, France).

Treatment doses—Bacteria: 2×10⁸ in 200 μL. The a-CTLA-4 was injected at 10 mg/kg/inj. Anti-CTLA-4 was administered at a dose volume of 10 mL/kg/adm (i.e. for one mouse weighing 20 g, 200 μL of test substance will be administered) according to the most recent body weight of mice.

Routes of administration—Bacterial inoculum was administered by oral gavage (per os, PO) via a cannula. Cannulas were decontaminated every day. Anti-CTLA-4 was injected into the peritoneal cavity of mice (Intraperitoneally, IP).

Culture conditions of bacterial strain—The culture conditions for the bacterial strain were as follows:
Pipette 10 mL of YCFA (from the prepared 10 mL E&O lab bottles) into Hungate tubes
Seal the tubes and flush with CO₂ using a syringe input and exhaust system
Autoclave the Hungate tubes
When cooled, inoculate the Hungate tubes with 1 mL of the glycerol stocks
Place the tubes in a static 37° C. incubator for about 16 hours.
The following day, take 1 mL of this subculture and inoculate 10 mL of YCFA (pre-warmed flushed Hungate tubes again, all in duplicate)
Place them in a static 37° C. incubator for 5 to 6 h

Cancer Cell Line and Culture Conditions—

The cell lines that were used are detailed in the table below:

| Cell line | Type | Mouse strain | Origin |
|---|---|---|---|
| EMT-6 | Breast carcinoma | BALB/c | ATCC |
| LL/2 (LLC1) | Lung carcinoma | C57BL/6 | ATCC CRL1642 |
| Hepal-6 | Hepatocellular | C57BL/6 | IPSEN |
| | carcinoma | | INNOVATION |
| RENCA | Renal adenocarcinoma | BALB/c | ATCC |

The EMT-6 cell line was established from a transplantable murine mammary carcinoma that arose in a BALB/cCRGL mouse after implantation of a hyperplastic mammary alveolar nodule [47].

The LL/2 (LLC1) cell line was established from the lung of a C57BL mouse bearing a tumor resulting from an implantation of primary Lewis lung carcinoma [48].

The Hepa 1-6 cell line is a derivative of the BW7756 mouse hepatoma that arose in a C57/L mouse [49].

Cell culture conditions—All cell lines were grown as monolayer at 37° C. in a humidified atmosphere (5% CO₂, 95% air). The culture medium and supplement are indicated in the table below:

| Cell | | |
|---|---|---|
| line | Culture medium | Supplement |
| EMT6 | RPMI 1640 containing 2 | 10% fetal bovine serum |
| | mM L-glutamine (ref: BE12- | (ref: #3302, Lonza) |
| | 702F, Lonza) | |
| LL/2 | RPMI 1640 containing 2 | 10% fetal bovine serum |
| (LLC1) | mM L-glutamine (ref: BE12- | (ref: #3302, Lonza) |
| | 702F, Lonza) | |
| Hepal-6 | DMEM (ref: 11960-044, | 10% fetal bovine serum |
| | Gibco) | (ref: #3302, Lonza) |
| | | 2 mM L-Glutamine |
| | | penicillin-streptomycin |
| | | (Sigma G-6784) |
| RENCA | DMEM | 10% fetal bovine serum, |
| | | 2 mM L-glutamine, 1 ug/ml |
| | | puromycin |

For experimental use, adherent tumor cells were detached from the culture flask by a 5 minute treatment with trypsin-versene (ref: BE17-161E, Lonza), in Hanks' medium without calcium or magnesium (ref: BE10-543F, Lonza) and neutralized by addition of complete culture medium. The cells were counted in a hemocytometer and their viability will be assessed by 0.25% trypan blue exclusion assay.

Use of Animals—

Healthy female Balb/C (BALB/cByJ) mice, of matching weight and age, were obtained from CHARLES RIVER (L'Arbresles) for the EMT6 and RENCA model experiments.

Healthy female C57BL/6 (C57BL16J) mice, of matching weight and age, were obtained from CHARLES RIVER (L'Arbresles) for the LL/2(LLC1) and the Hepa1-6 model experiments.

Animals were maintained in SPF health status according to the FELASA guidelines, and animal housing and experimental procedures according to the French and European Regulations and NRC Guide for the Care and Use of Laboratory Animals were followed [50,51]. Animals were maintained in housing rooms under controlled environmental conditions: Temperature: 22±2° C., Humidity 55±10%, Photoperiod (12 h light/12 h dark), HEPA filtered air, 15 air exchanges per hour with no recirculation. Animal enclosures were provided with sterile and adequate space with bedding material, food and water, environmental and social enrichment (group housing) as described: 900 cm² cages (ref: green, Tecniplast) in ventilated racks, Epicea bedding (SAFE), 10 kGy Irradiated diet (A04-10, SAFE), Complete food for immuno-competent rodents—R/M-H Extrudate, water from water bottles.

Experimental Design and Treatments

Antitumor Activity, EMT6 Model

Treatment schedule—The start of first dosing was considered as D0. On D0, non-engrafted mice were randomized according to their individual body weight into groups of 9/8 using Vivo Manager® software (Biosystemes, Couternon, France). On D0, the mice received vehicle (culture medium) or bacterial strain. On D14, all mice were engrafted with EMT-6 tumor cells as described below. On D24, mice from the positive control group received anti-CTLA-4 antibody treatments.

The treatment schedule is summarized in the table below:

| | | | | | Treatment |
|---|---|---|---|---|---|
| Group | No. Animals | Treatment | Dose | Route | Schedule |
| 1 | 8 | Untreated | — | — | — |
| 2 | 8 | Vehicle (media) | — | PO | Q1D × 42 |
| 3 | 9 | Bacterial strain # 1 | 2 × 108 | PO | Q1D × 42 |
| | | (MRX518) | bacteria | | |
| 4 | 8 | Anti-CTLA4 | 10 mg/kg | IP | TW × 2 |

The monitoring of animals was performed as described below.

Induction of EMT6 tumors in animals—On D14, tumors were induced by subcutaneous injection of 1×10⁶ EMT-6 cells in 200 μL RPMI 1640 into the right flank of mice.

Euthanasia—Each mouse was euthanized when it reached a humane endpoint as described below, or after a maximum of 6 weeks post start of dosing.

Antitumor Activity, LL/2 (LLC1) Model

Treatment schedule—The start of first dosing was considered as D0. On D0, non-engrafted mice were randomized according to their individual body weight into 7 groups of 9/8 using Vivo Manager® software (Biosystemes, Couternon, France). On D0, the mice will received vehicle (culture medium) or bacterial strain. On D14, all mice were engrafted with LL/2 tumor cells as described below. On D27, mice from the positive control group received anti-CTLA-4 antibody treatments.

The treatment schedule is summarized in the table below:

| | | | | | Treatment |
|---|---|---|---|---|---|
| Group | No. Animals | Treatment | Dose | Route | Schedule |
| 1 | 8 | Untreated | — | — | — |
| 2 | 9 | Vehicle (media) | — | PO | Q1D × 42 |
| 3 | 9 | Bacterial strain # 1 | 2 × 10⁸ | PO | Q1D × 42 |
| | | (MRX518) | bacteria | | |
| 4 | 8 | Anti-CTLA4 | 10 mg/kg | IP | TW × 2 |

The monitoring of animals was performed as described below.

Induction of LL/2 (LLC1) tumors in animals—On D14, tumors were induced by subcutaneous injection of 1×10⁶ LL/2 (LLC1) cells in 200 μL RPMI 1640 into the right flank of mice.

Euthanasia—Each mouse was euthanized when it reached a humane endpoint as described below, or after a maximum of 6 weeks post start of dosing.

Antitumor Activity, Hepa1-6 Model

Treatment schedule—The start of first dosing was considered as D0. On D0, non-engrafted mice were randomized according to their individual body weight into 7 groups of 9 using Vivo Manager® software (Biosystemes, Couternon, France). On D0, the mice received vehicle (culture medium) or bacterial strain. On D14, all mice were engrafted with Hepa 1-6 tumor cells as described below. On D16, mice from the positive control group received anti-CTLA-4 antibody treatments.

The treatment schedule is summarized in the table below:

| | | | | | Treatment |
|---|---|---|---|---|---|
| Group | No. Animals | Treatment | Dose | Route | Schedule |
| 1 | 9 | Untreated | — | — | — |
| 2 | 9 | Vehicle (media) | — | PO | Q1D × 42 |
| 6 | 9 | Bacterial strain #4 | 2 × 10⁸ | PO | Q1D × 42 |
| | | (MRX518) | bacteria | | |
| 7 | 9 | Anti-CTLA4 | 10 mg/kg | IP | TW × 2 |

The monitoring of animals was performed as described below.

Orthotopic induction of Hepa 1-6 tumor cells in animals by intrasplenic injection—On D14, one million (1×10⁶) Hepa 1-6 tumor cells in 50 μL RPMI 1640 medium were transplanted via intra-splenic injection into mice. Briefly, a small left subcostal flank incision was made and the spleen was exteriorized. The spleen was exposed on a sterile gauze pad, and injected under visual control with the cell suspension with a 27-gauge needle. After the cell inoculation, the spleen was excised.

Euthanasia—Each mouse was euthanized when it reached a humane endpoint as described in section below, or after a maximum of 6 weeks post start of dosing.

Evaluation of tumor burden at euthanasia—At the time of termination, livers were collected and weighed.

Antitumor Activity, RENCA Model

Treatment schedule—The start of first dosing was considered as D0. On D0, non-engrafted mice were randomized according to their individual body weight into groups of 9 mice using Vivo Manager® software (Biosystemes, Couternon, France). On D0, the mice received vehicle (culture medium) or bacterial strain (2×10⁸ in 200 μL, PO). On D14, all mice were engrafted with RENCA tumour cells injected SC into the ventral surface of the lower flank as described below. Treatment with anti-CTLA-4 (10 mg/kg, IP) and anti-PDL1 (clone 10F.9G2, 10 mg/kg) was initiated when tumours reached a volume of 50-70 mm3.

The treatment schedule is summarized in the table below:

| | | | | | Treatment |
|---|---|---|---|---|---|
| Group | No. Animals | Treatment | Dose | Route | Schedule |
| 1 | 9 | Untreated | — | — | — |
| 2 | 9 | Vehicle (media) | — | PO | Q1D × 42 |
| 3 | 9 | Bacterial strain | 2 × 10⁸ | PO | Q1D × 42 |
| | | (MRX518) | bacteria | | |
| 4 | 9 | Paclitaxel | 15 mg/kg | IP | Q4D (every |
| | | | | | four days) |
| 5 | 9 | Anti-CTLA4 + | 10 mg/kg + | IP | TW × 2 |
| | | Anti-PDL1 | 10 mg/kg | | |

The monitoring of animals was performed as described below.

Orthotopic induction of RENCA tumor cells in animals by SC injection—On D14, one million (1×10⁶) RENCA tumor cells in 50 μL RPMI 1640 medium were transplanted via SC injection into the ventral surface of the lower flank of mice.

Euthanasia—Each mouse was euthanized when it reached a humane endpoint as described in section below, or after a maximum of 6 weeks post start of dosing.

Evaluation of tumour burden at euthanasia—At the time of termination, tumours were collected and their volume evaluated.

Animal Monitoring

Clinical monitoring—The length and width of the tumour was measured twice a week with callipers and the volume of the tumour was estimated by this formula [52]:

$$Tumor volume = \frac{{width}^{2} \times length}{2}$$

Humane endpoints [53]: Signs of pain, suffering or distress: pain posture, pain face mask, behaviour; Tumor exceeding 10% of normal body weight, but non-exceeding 2000 mm³; Tumors interfering with ambulation or nutrition; Ulcerated tumor or tissue erosion; 20% body weight loss remaining for 3 consecutive days; Poor body condition, emaciation, cachexia, dehydration; Prolonged absence of voluntary responses to external stimuli; Rapid laboured breathing, anaemia, significant bleeding; Neurologic signs: circling, convulsion, paralysis; Sustained decrease in body temperature; Abdominal distension.

Anaesthesia—Isoflurane gas anesthesia were used for all procedures: surgery or tumor inoculation, i.v. injections, blood collection. Ketamine and Xylazine anesthesia were used for stereotaxia surgical procedure.

Analgesia—Carprofen or multimodal carprofen/buprenorphine analgesia protocol were adapted to the severity of surgical procedure. Non-pharmacological care was provided for all painful procedures.

Additionally, pharmacological care not interfering with studies (topic treatment) were provided at the recommendation of the attending veterinarian.

Euthanasia—Euthanasia of animals was performed by gas anesthesia over-dosage (Isoflurane) followed by cervical dislocation or exsanguination.

Results

Antitumor Activity, EMT6 Model

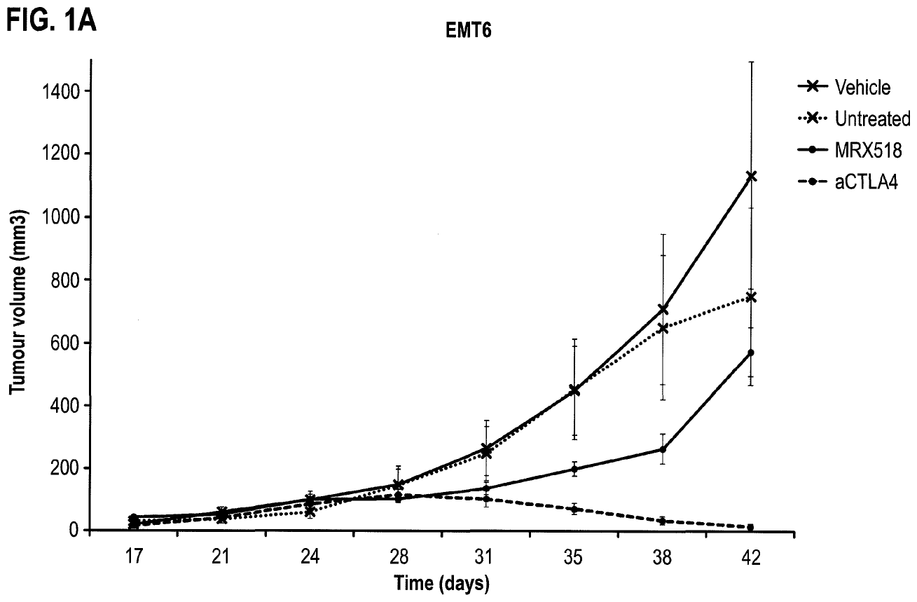

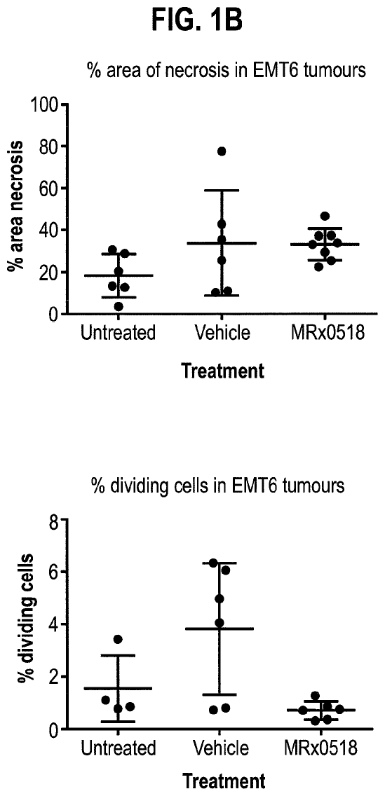

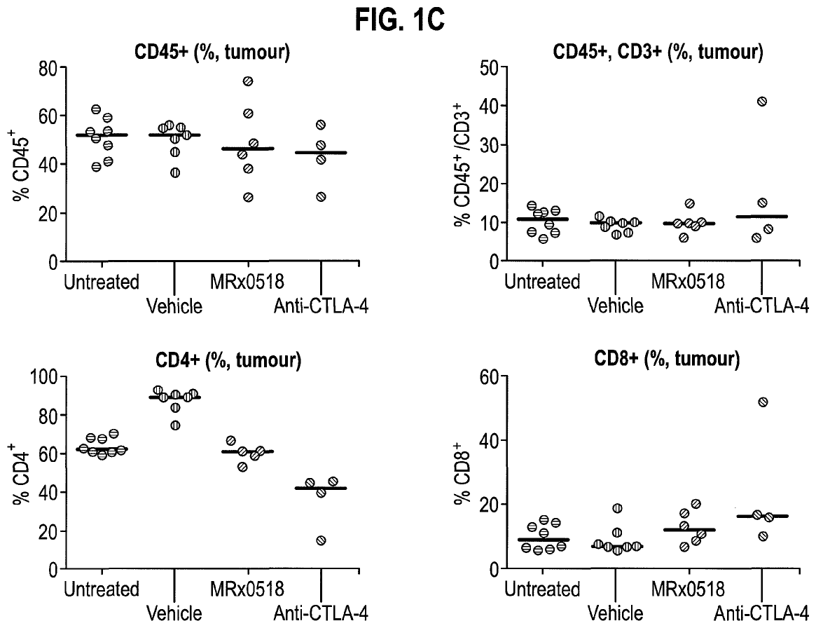

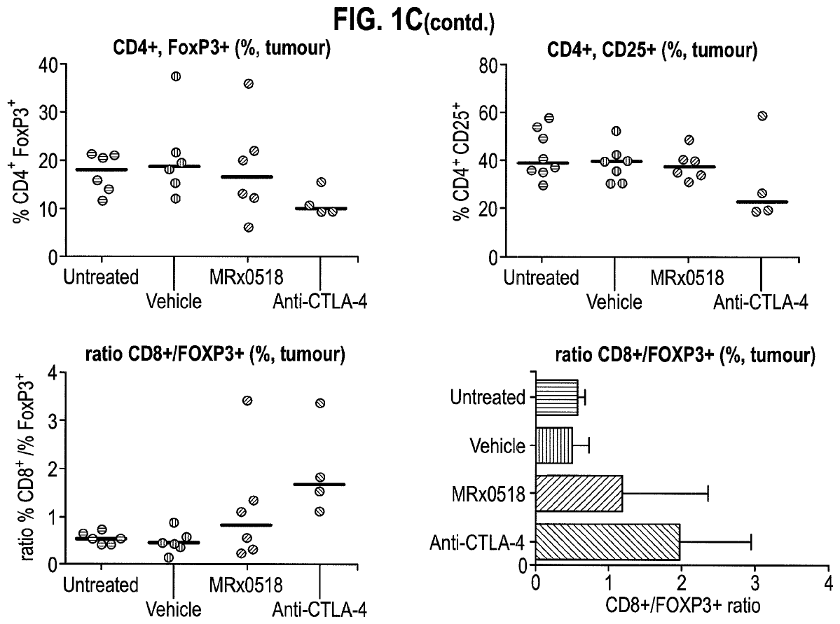

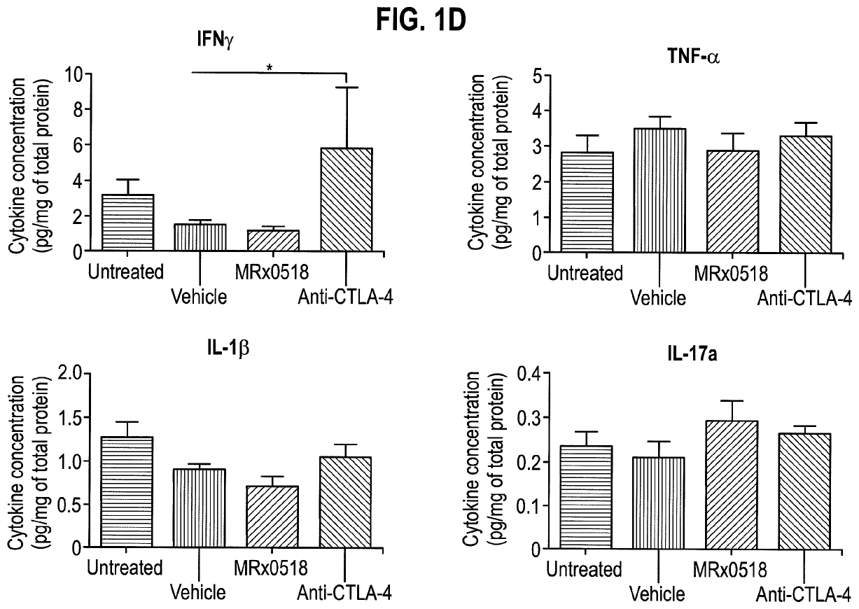

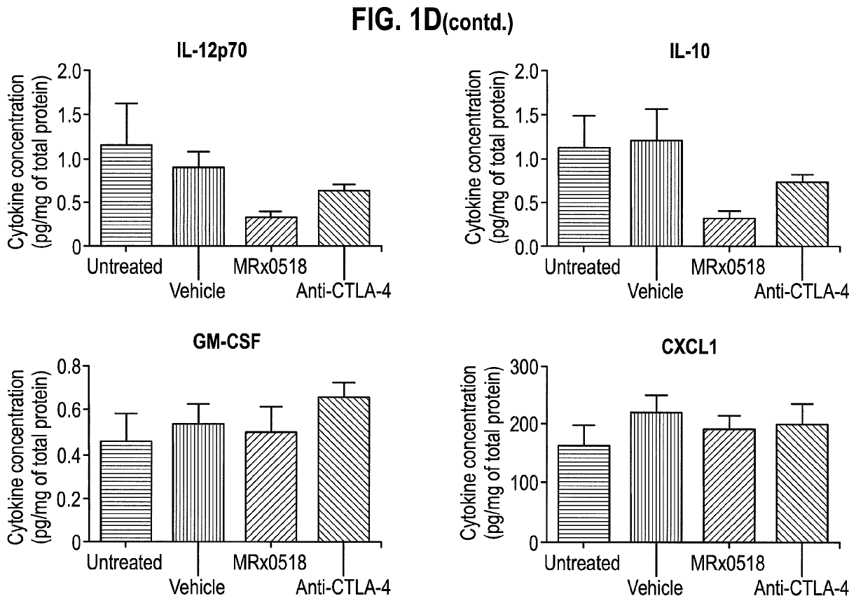

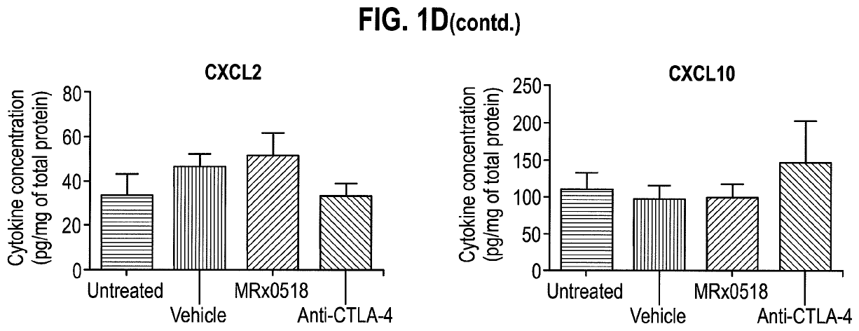

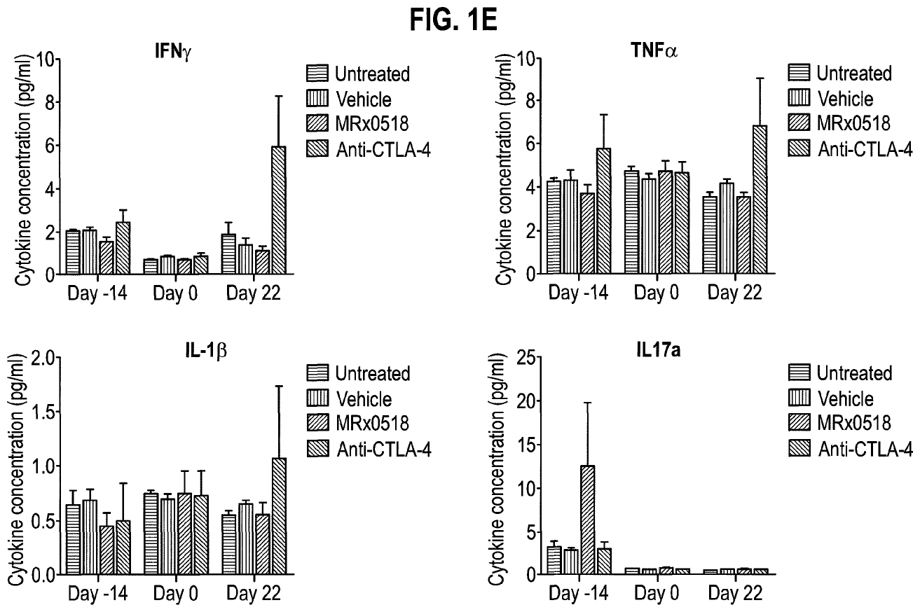

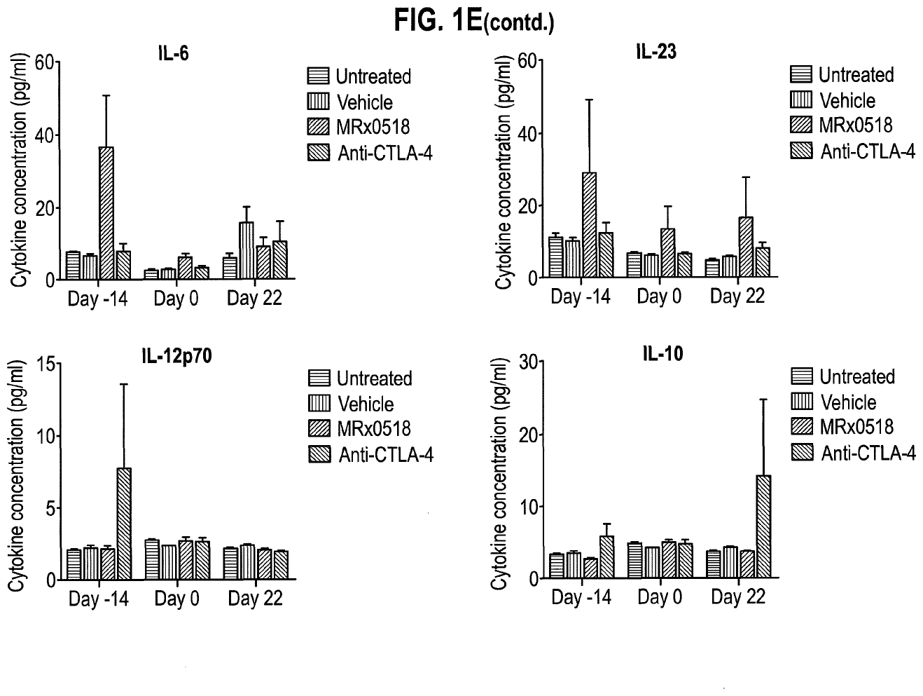

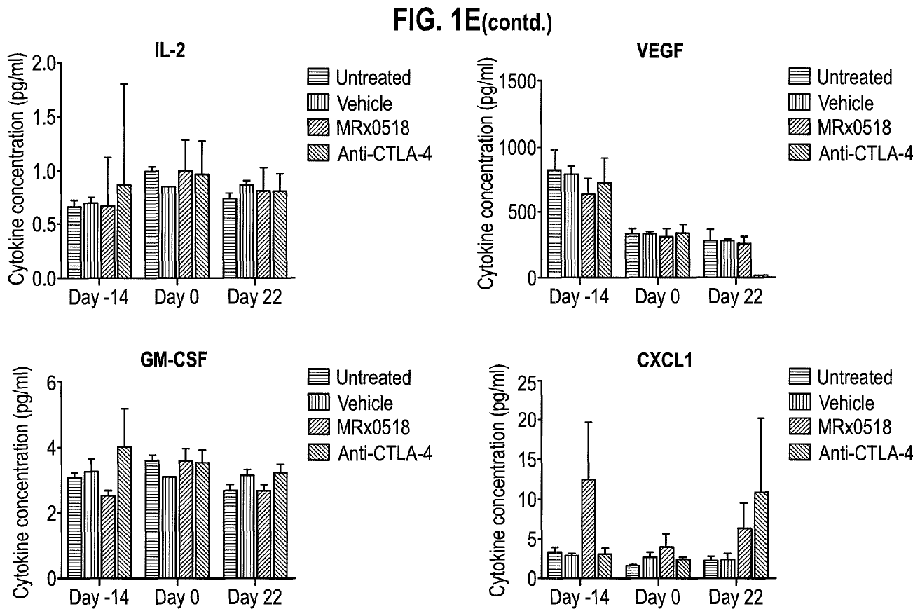

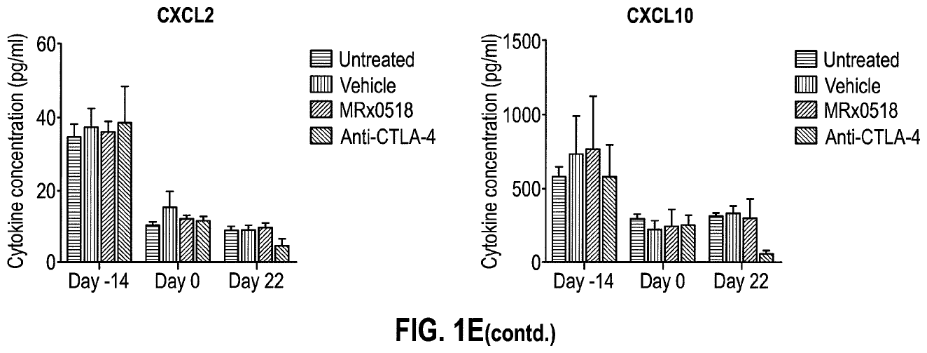

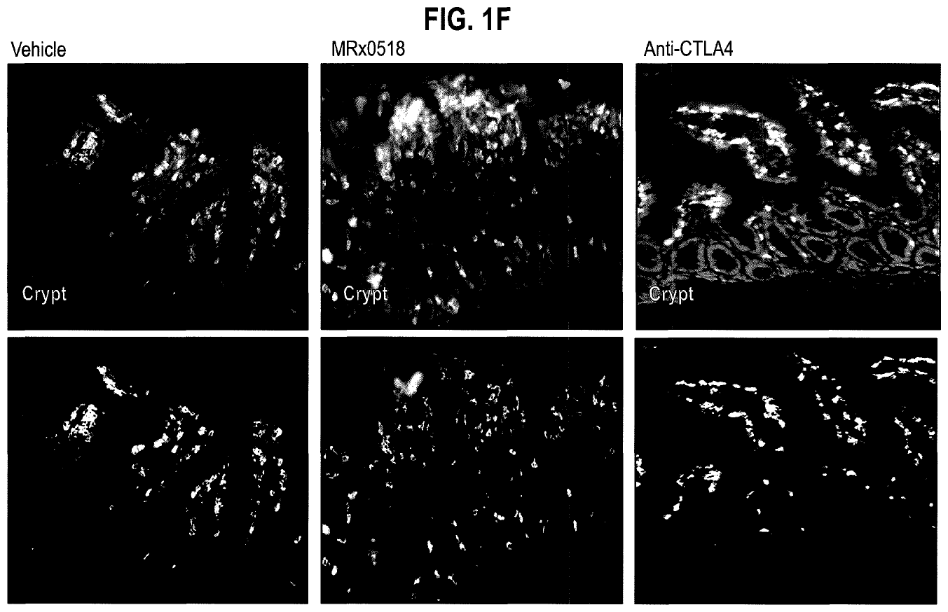

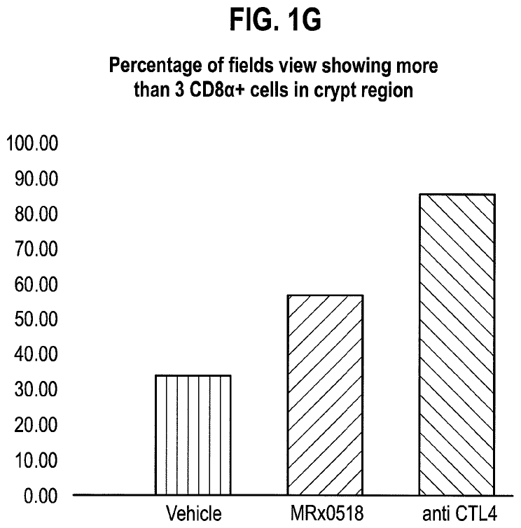

The results are shown in FIG. 1A. Treatment with the bacterial strain of the invention led to a clear reduction in tumour volume relative to both the negative controls. The positive control also led to a reduction in tumour volume, as would be expected.

To further elucidate the mechanisms through which MRx0518 conveys its therapeutic effects in syngeneic tumour models, ex vivo analysis was performed on the syngeneic EMT6 tumour model studies. While tumour volume is the primary measurement in preclinical oncology studies, tumours often consist of actively dividing tumour cells along with a necrotic core. To investigate whether MRx0518 treatment had influence on the degree of necrosis found within EMT6 tumours, paraffin sections from the mid-belly region of the tumours were stained with Haematoxylin and Eosin. MRx0518 treatment of a murine EMT6 breast carcinoma model showed a tendency towards increasing the cross-sectional area of necrosis within the tumour (FIG. 1B, upper panel). To investigate whether MRx0518 treatment had influence on dividing cells within the tumour, paraffin sections from the mid-belly region of the tumours were stained with the proliferation protein Ki67, along with DAPI counter stain, to estimate the percentage of cells dividing within the EMT6 tumour. MRx0518 treatment of a murine EMT6 breast carcinoma model significantly decreased the percentage of dividing cells seen within the tumour (FIG. 1B, lower panel, P=0.019).

Immune Cell Populations

Further investigation of the tumour microenvironment was performed through flow cytometry of the tumour, to investigate the hypothesis that the MRx518 bacterial strain has the ability to regulate the immune system into inducing an anti-tumour effect. Tumours excised from the different treatment groups were cut into pieces. One piece was subjected to flow cytometry analysis. To assess the relative percentage of T lymphocytes, present within the tumours, the following markers were used: CD45, CD3, CD4, CD8, CD25 and FoxP3.

The flow cytometry data shows that the relative percentage of lymphocytes in tumours was slightly decreased in both the MRx0518 and anti-CTLA-4 treated groups, when compared respectively to vehicle or control animals (FIG. 1C). Likewise, the relative percentage of CD4+ cells appeared to be decreased in MRx0518 and anti-CTLA-4 treated animals, whilst the relative percentage of CD8+ cells followed an opposite trend in both groups, albeit with different magnitude. The relative percentage of CD4+FoxP3+ cells was lower in the anti-CTLA-4 treated group when compared to the slight decrease in MRx0518 treated animals; however, the reduction in the relative percentage of CD4+CD25+ cells was noticeable only in the anti-CTLA-4 treated group. The CD8+/FoxP3+ ratio showed a greater increase in the anti-CTLA-4 treated group than in the MRx0518 animals. These data presented here supports the hypothesis that anti-CTLA-4 antibody targets regulatory T cells (Tregs) by reducing their cell numbers or attenuating their suppressive activity in tumour tissue, whilst suggesting a different mode of action for MRx0518.

Cytokine Production

An additional tumour piece was used for total protein extraction and subsequent cytokine analysis, together with plasma samples. Protein levels of IL-10, CXCL1, CXCL2, CXCL10, IL-1B, IL-17A, GM-CSF, TNF-α, IL-12p70 and IFN-γ in the tumour microenvironment were analysed by MagPix technology. While IL-17A and GM-CSF were below levels of detection, all the other markers were expressed at reasonable levels (FIG. 1D). A significance difference was observed between the vehicle and anti-CTLA-4 group for IFN-γ. The production of the IL-10 and IL-12p70 immune markers seemed reduced following MRx518 treatment compared to the control treatments.

Cytokine levels were also assessed in blood plasma of the same animals. Protein levels of IL-23, IL-6, IL-10, VEGF, CXCL1, CXCL2, CXCL10, IL-2, IL-1B, IL-17A, GM-CSF, TNF-α, IL-12p70 and IFN-γ were analysed by MagPix technology. Overall, little cytokine production was detected in the blood plasma of animals either before tumour induction or at the end of the study (FIG. 1E). VEGF and CXCL10 were detected at substantial levels, while IL-23, IL-6, IL-10, CXCL1 and CXCL2 were detected at low levels. IL-2, IL-1b, IL-17A, GM-CSF, TNF-α, IL-12p70 and IFN-γ were not detected in the samples. MRx0518 significantly increased production of IL-6 at Day 0. MRx0518 also seemed to increase IL-23 production. VEGF and CXCL10 were significantly downregulated in the anti-CLTA-4 group at Day 22. Similarly to the results shown for the immune cell populations, the differences in cytokine production in the tumour and plasma, between MRx518 and CTLA-4 suggests that each of them acts on a distinct and potentially complementary mechanism.

Localisation of CD8α Positive Cells in the Ileum

10 μm cryo-sections of ileum were cut in cryostat (CM 1950 Leica), picked up onto poly-L Lysine slides. The sections were then air-dried for 1 hour, fixed for 10 minutes in ice-cold methanol, washed in PBS, blocked in 10% BSA in PBS pH 7.2 before being incubated overnight with the primary antibody (rat-anti-mouse-CD8α antibody, Sigma-Aldrich, Millipore).

The next morning the slides were washed in PBS and stained with a secondary antibody: goat-anti-rat-antibody-Alexa488 (Molecular Probe, Invitrogen) for 1 hour at room temperature. After another washing step, the slides were counterstained with 4′,6-diamidino-2-phenylindole dihydrochloride (DAPI) (Sigma-Aldrich, Millipore) and mounted in Vectashield (Vector Laboratories). The slides were viewed and imaged using a Zeiss Axioscope Microscope equipped with a mercury vapour lamp, appropriate filters and a x20 apochromatic objective. Examples of images obtained from slides from the vehicle, MRx0518, and anti CTL4 animals are shown (FIG. 1F—upper panels: DAPI staining, lower panels: CD8α staining).

Fields of view were examined from 20 animals and imaged using manual exposure time. The number of animals and fields analysed are shown in the following table:

| | Number of fields | Number of |
|---|---|---|
| Group | analysed | mice |
| Vehicle | 53 | 5 |
| MRx0518 | 70 | 7 |
| anti | 71 | 8 |
| CTL4 | | |

The images were scored as follow: fields with <3 positive cells were scored as 0, whilst fields with more >3 cells were scored as 1. The results of this analysis are shown (FIG. 1G).

Ileum cryosections stained with anti-CD8α showed a higher number of CD8α positive cells localized in the crypt region tissues from animals treated with MRx0518 and anti-CTLA-4 compared to the vehicle group.

This observation is in line with CD8+ T cells being present in the intestine in case of infection or inflammatory microenvironment, as part of the immune response.

Antitumor Activity, LL/2 (LLC1) Model

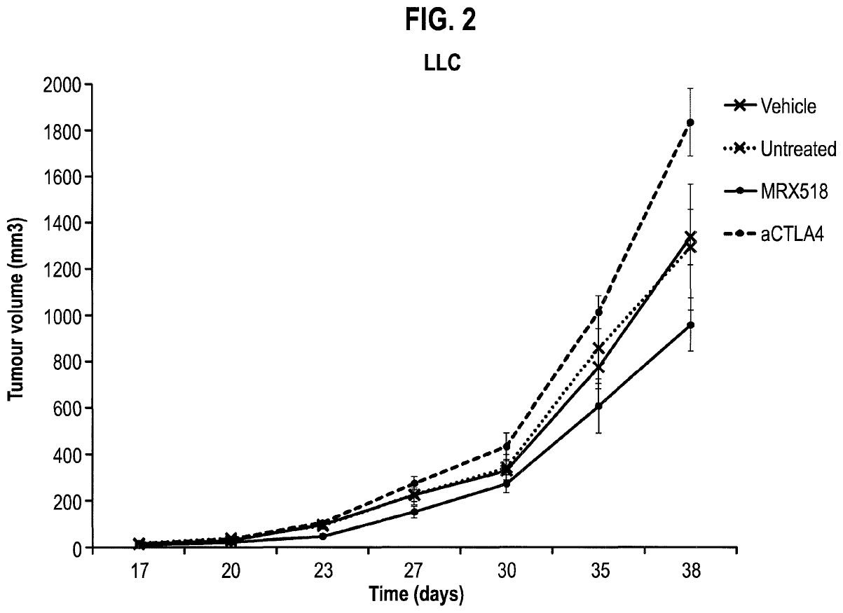

The results are shown in FIG. 2. Treatment with the bacterial strain of the invention led to a clear reduction in tumour volume relative to both the negative controls.

Antitumor Activity, Hepa1-6 Model

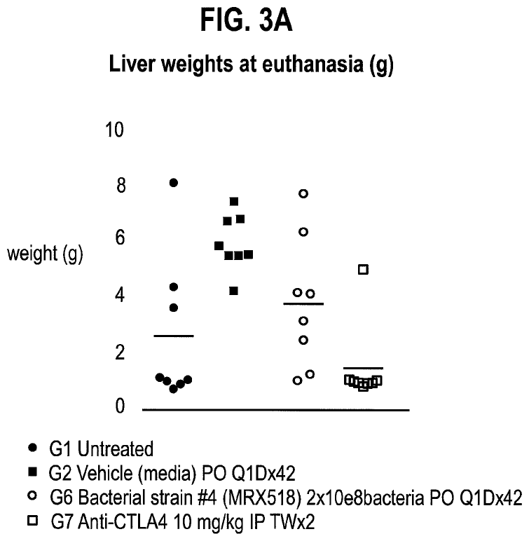

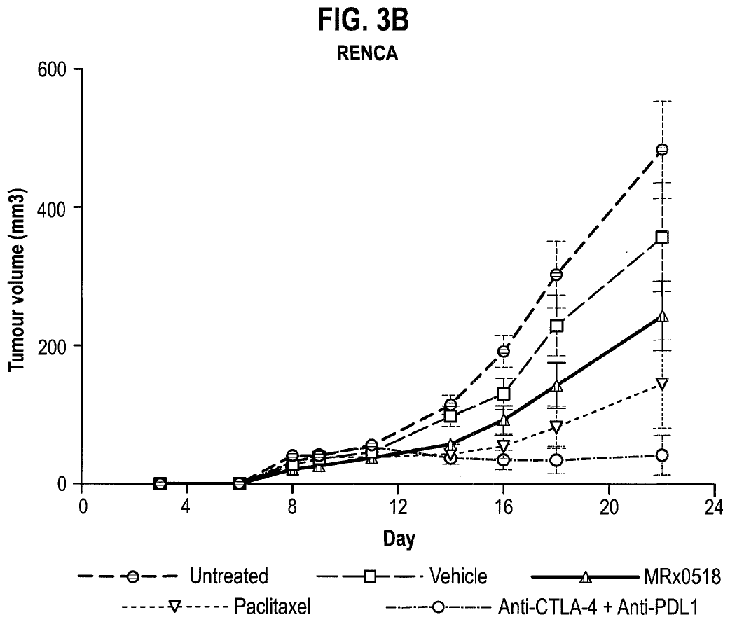

The results are shown in FIG. 3A. The untreated negative control does not appear as would be expected, because liver weight was lower in this group than the other groups. However, the vehicle negative control and the positive control groups both appear as would be expected, because mice treated with vehicle alone had larger livers than mice treated with anti-CTLA4 antibodies, reflecting a greater tumour burden in the vehicle negative control group. Treatment with the bacterial strain of the invention led to a clear reduction in liver weight (and therefore tumour burden) relative to the mice in the vehicle negative control group.

Antitumor Activity, RENCA Model

The results are shown in FIG. 3B. Treatment with MRx0518 monotherapy reduced tumour volume with Test/Control of 51% (day 18) compared with the vehicle-treated groups. Paclitaxel and anti-CTLA-4+anti-PDL-1 showed an (almost) complete reduction in tumour size at D18 and D22 compared to both the untreated and vehicle groups.

These data indicate that strain MRX518 may be useful for treating or preventing cancer, and in particular for reducing tumour volume in breast, lung, kidney and liver cancers.

### Example 2—PCR Gene Analysis

A pure culture of bacteria MRX518 was studied in a PCR gene analysis. There were two arms to the experiment: 1) MRX518 was co-cultured with human colonic cells (CaCo2) to investigate the effects of the bacteria on the host, and 2) MRX518 was co-cultured on CaCo2 cells that were stimulated with IL1 to mimic the effect of the bacteria in an inflammatory environment. The effects in both scenarios were evaluated through gene expression analysis. The results are shown below:

| Gene | Fold change | Function |
|---|---|---|
| CXCL3 | 28412.73 | CXCR2 ligand, |
| CXCL2 | 135.42 | CXCR2 ligand, 90% homology with |
| | | CXCL1. |
| CXCL9 | 34.76 | CXCR3 ligand, primarily thought of as Th1 |
| | | cell chemoattractant (inducible by IFN-g) |
| IL8 | 31.81 | Cytokine, chemoattractant (especially |
| | | neutrophils), many receptors including |
| | | CXCR1 and CXCR2/ |
| CXCL1 | 16.48 | CXCR2 ligand, stimulates cell proliferation |
| | | as well as migration, overexpression is |
| | | neuroprotective in EAE. |
| CD40 | 14.33 | Co-stimulatory molecule, route of T cell |
| | | dependent DC activation. |
| TNF | 13.50 | Major proinflammatory cytokine |
| IL17C | 12.18 | Promotes antibacterial response from |
| | | epthielium, synergistic with IL-22, |
| CXCL10 | 10.66 | Close homology with CXCL9, think also |
| | | CXCR3 ligand? |
| HSPA1B | 10.19 | Heat shock protein |
| NFKBIA | 8.87 | NFkB signalling; PI3K |
| JUN | 7.61 | Antibacterial response; GPCR signalling. |
| TNFAIP3 | 6.63 | TNF signalling |
| DUSP1 | 6.36 | Anti-inflammatory phosphatase, inactivates |
| | | MAPKs |
| JUNB | 5.36 | Transcription factor, JAK-STAT signalling |
| BIRC3 | 4.86 | Adherens junctions, tight junctions |
| DUSP2 | 4.59 | Anti-inflammatory, inactivates MAPK. |
| IL32 | 4.29 | Proinflammatory cytokine, induced by |
| | | IFN-g, IL-18 |
| DUSP5 | 3.12 | Anti-inflammatory, inactivates MAPK |
| FOS | 3.03 | Transcription factors, TLR signalling, |
| | | forms part of AP-1 |
| GADD45B | 2.89 | Cell growth and proliferation |
| CLDN4 | 2.61 | Tight junctions |
| ADM | 2.57 | NFkB signalling |
| KLF10 | 2.49 | Cell arrest, TGF-b singllaing. |
| DEFB4A | −2.34 | Antimicrobial peptide |
| APBA1 | −2.53 | Signalling |
| IGFBP1 | −2.72 | Signalling pathway |
| IL28B | −2.73 | IFN-lambda, antiviral immune defence, |
| IL10 | −3.38 | Anti-inflammatory cytokine |
| NR4A1 | −5.57 | Nuclear receptor, anti-inflammatory, |
| | | regulator of T cell proliferation. T helper |
| | | cell differentiation |
| NOD2 | −14.98 | PRR, inflammasome activator, promotes |
| | | autophagy |
| INOS | −26.88 | Proinflammatory, generator of nitric oxide |

These data appear to show two gene expression signatures—CXCR1/2 ligands (CXCL3, CXCL2, CXCL1, IL-8), which is associated with pro-inflammatory cell migration, and CXCR3 ligands (CXCL9, CXCL10), which is more specifically indicative of IFN-γ-type responses, also supported by IL-32, which is IFN-γ-inducible.

### Example 3—Stability Testing

A composition described herein containing at least one bacterial strain described herein is stored in a sealed container at 25° C. or 4° C. and the container is placed in an atmosphere having 30%, 40%, 50%, 60%, 70%, 75%, 80%, 90% or 95% relative humidity. After 1 month, 2 months, 3 months, 6 months, 1 year, 1.5 years, 2 years, 2.5 years or 3 years, at least 50%, 60%, 70%, 80% or 90% of the bacterial strain shall remain as measured in colony forming units determined by standard protocols.

### Example 4—Cytokine Production in Immature Dendritic Cells Induced by MRX518 Compared to MRX518+LPS

Summary

This study tested the effect of the bacterial strain MRX518 alone and in combination with lipopolysaccharide (LPS) on cytokine production in immature dendritic cells.

A monocyte population was isolated from peripheral blood mononuclear cells (PBMCs). The monocyte cells were subsequently differentiated into immature dendritic cells. The immature dendritic cells were plated out at 200,000 cells/well and incubated with MRX518 at a final concentration of 10⁷/ml, with the optional addition of LPS at a final concentration of 100 ng/ml. The negative control involved incubating the cells with RPMI media alone and positive controls incubated the cells with LPS at a final concentration of 100 ng/ml. The cytokine content of the cells was then analysed.

Results

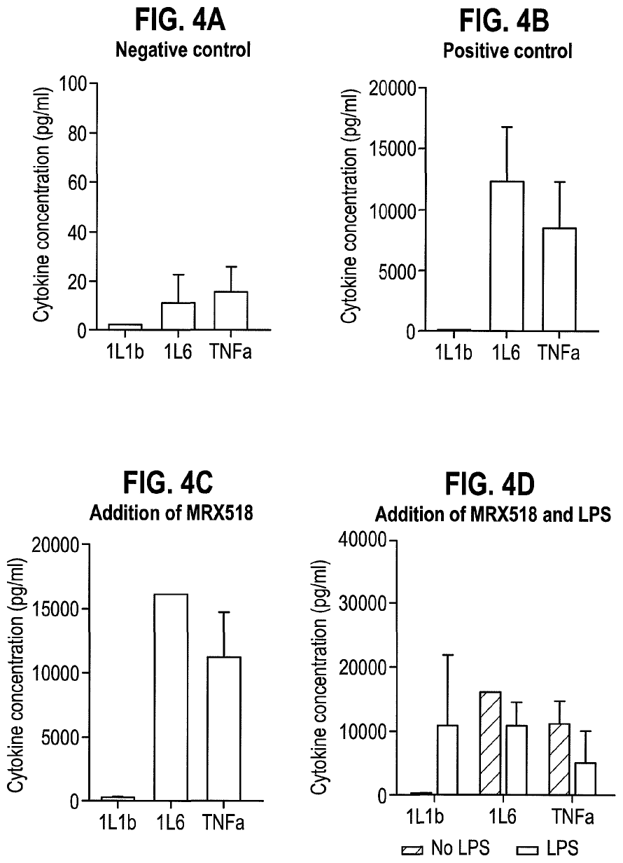

The results of these experiments can be seen in FIGS. 4a-d. The addition of MRX518 alone leads to a substantial increase in the level of cytokines IL-6 and TNF-α compared to the negative control (FIGS. 4a and c). The addition of LPS (positive control) leads to an increase in the level of IL-6 and TNF-α compared to the negative control but not IL-1β (FIG. 4b). A combination of MRX518 and LPS led to a synergistic increase in the level of IL-1β produced (FIG. 4d).

Conclusion

MRX518 has the ability to induce higher IL-6 and TNF-α cytokine production in immature dendritic cells. The combination LPS and MRX518 can increase the levels of cytokines IL-1β in immature dendritic cells. These data indicate that MRX518 alone or in combination with LPS can increase inflammatory cytokines IL-1β, IL-6 and TNF-α, which promotes inflammation that can suppress cancer. Treatment with MRX518 alone or in combination with can induce cytokines that can limit tumour growth.

### Example 5—Cytokine Production in THP-1 Cells Induced by MRX518 Compared to MRX518+LPS

Summary

This study tested the effect of bacterial strain MRX518 alone and in combination with LPS on cytokine production in THP-1 cells, a model cell line for monocytes and macrophages.

THF-1 cells were differentiated into MO medium for 48 h with 5 ng/mL phorbol-12-myristate-13-acetate (PMA). These cells were subsequently incubated with MRX518 at a final concentration of 10⁸/ml, with or without the addition of LPS at a final concentration of 100 ng/ml. The bacteria were then washed off and the cells allowed to incubate under normal growing conditions for 24 h. The cells were then spun down and the resulting supernatant was analysed for cytokine content.

Results

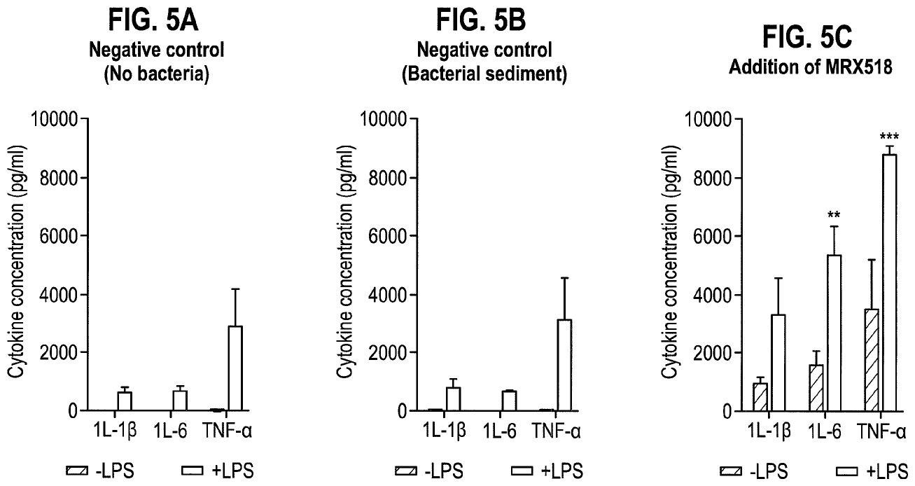

The results of these experiments can be seen in FIGS. 5a-c. The addition of MRX518 without LPS leads to an increase in the cytokine levels of IL-1β, IL-6 and TNF-α compared to the no bacterial and the bacterial sediment controls. The addition of LPS and MRX518 leads to a synergistic increase in the production of cytokines.

Conclusion

MRX518 has the ability to induce cytokine production in THP-1 cells, which can be synergistically increased with the addition of LPS. These data indicate that MRX518 alone or in combination with LPS can increase inflammatory cytokines IL-1β, IL-6 and TNF-α, which promotes inflammation that can suppress cancer. Treatment with MRX518 alone or in combination with can induce cytokines that can limit tumour growth.

### Example 6—Antitumour Activity of a Therapeutic Combination of MRX518 and a PD-1 Inhibitor or a CTLA-4 Inhibitor

Summary

This study compared the anti-tumour activity of MRX518, a PD-1 inhibitor, a CTLA-4 inhibitor and therapeutic combinations of MRX518 with the PD-1 inhibitor or the CTLA-4 inhibitor in mice bearing EMT-6 tumour cells.

Materials

Test and reference substances—Bacterial strain #MRX518; Anti-PD-1 antibody (clone: RMP1-14, catalog: BE0146, isotype: Rat IgG2a, Bioxcell); Anti-CTLA4 antibody (ref: BE0131, Bioxcell; clone: 9H10; reactivity: mouse; isotype: Hamster IgG1; storage conditions: +4° C.).

Test and reference substances vehicles—The MRX518 bacteria were grown in a bacterial culture medium (Yeast extract, Casitone, Fatty Acid medium (YCFA)) and kept as a glycerol stock at −80° C. The animals were dosed with the bacteria according to the study protocol. The anti-PD1 and anti-CTLA-4 antibodies were diluted with PBS (ref: BE14-516F, Lonza, France) on each day of injection to mice.

Treatment doses—Bacteria: 2×10⁸ in 200 μL. The anti PD1-1 and anti CTLA4 antibodies were administered at 10 mg/kg body weight according to the most recent body weight of mice.

Routes of administration—The bacterial composition was administered by oral gavage (per os, PO) via a gavage tube at a volume of 200 μL/inj. The anti PD-1 and anti CTLA-4 antibodies were injected into the peritoneal cavity of mice (Intraperitoneally, IP) at a volume of 10 ml/kg adjusted to the most recent individual body weight of mice.

Cancer cell line and culture conditions—The cell line that was used in this study is the EMT-6 cell line that was obtained from the ATCC (American Type Culture Collection, Manassas, Va., USA). The EMT-6 cell line was established from a transplantable murine mammary carcinoma that arose in a BALB/cCRGL mouse after implantation of a hyperplastic mammary alveolar nodule.

Tumor cells were grown as monolayer at 37° C. in a humidified atmosphere (5% CO2, 95% air). The culture medium was RPMI 1640 containing 2 mM L-glutamine (ref: BE12-702F, Lonza, Verviers, Belgium) supplemented with 10% fetal bovine serum (ref: 3302, Lonza). EMT-6 tumor cells are adherent to plastic flasks. For experimental use, tumor cells were detached from the culture flask by a 5-minute treatment with trypsin-versene (ref: BE02-007E, Lonza), in Hanks' medium without calcium or magnesium (ref: BE10-543F, Lonza) and neutralized by addition of complete culture medium. The cells were counted and their viability was assessed by 0.25% trypan blue exclusion assay.

Use of animals—One hundred and thirty (130) healthy female Balb/C (BALB/cByJ) mice, 5-7 weeks old, were obtained from CHARLES RIVER (L'Arbresles) and maintained in SPF health status according to the FELASA guidelines. Animal housing and experimental procedures were realized according to the French and European Regulations and NRC Guide for the Care and Use of Laboratory Animals. Animals were maintained 3-4 per cage in housing rooms under controlled environmental conditions: Temperature: 22±2° C., Humidity 55±10%, Photoperiod (12 h light/12 h dark), HEPA filtered air, 15 air exchanges per hour with no recirculation Animal enclosures were provided with sterile and adequate space with bedding material, food and water, environmental and social enrichment (group housing) as described: Top filter polycarbonate Eurostandard Type III or IV cages, Corn cob bedding (ref: LAB COB 12, SERLAB, France), 25 kGy Irradiated diet (Ssniff® Soest, Germany), Complete food for immunocompetent rodents—R/M-H Extrudate, Sterile, filtrated at 0.2 μm water and Environmental enrichment (SIZZLE-dri kraft—D20004 SERLAB, France). Animals are individually identified with RFID transponder and each cage was ladled with a specific code. Treatment of the animals started after one week of acclimation for batches 2 and 3, or after three weeks of acclimation for batch 1.

Experimental Design and Treatments

On day −14 (D-14), 130 non-engrafted mice were randomized according to their individual body weight into 3 groups of 30 animals and 4 groups of 10 animals using Vivo Manager® software (Biosystemes, Couternon, France). The mice were separated into 3 batches of 10 animals per treatment group (batch 1: 10 animals of groups 1, 2 and 3; batch 2: 10 animals of groups 1, 2 and 3 and batch 3: 10 animals of groups 1 to 7) with different termination points from the start of the study: D-14 or D0.

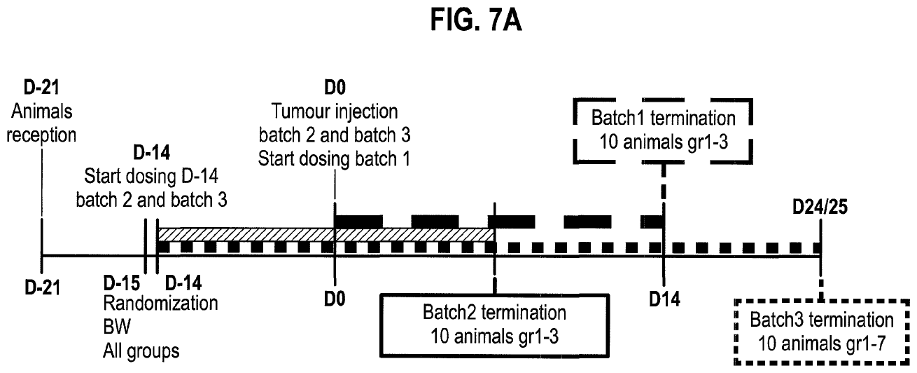

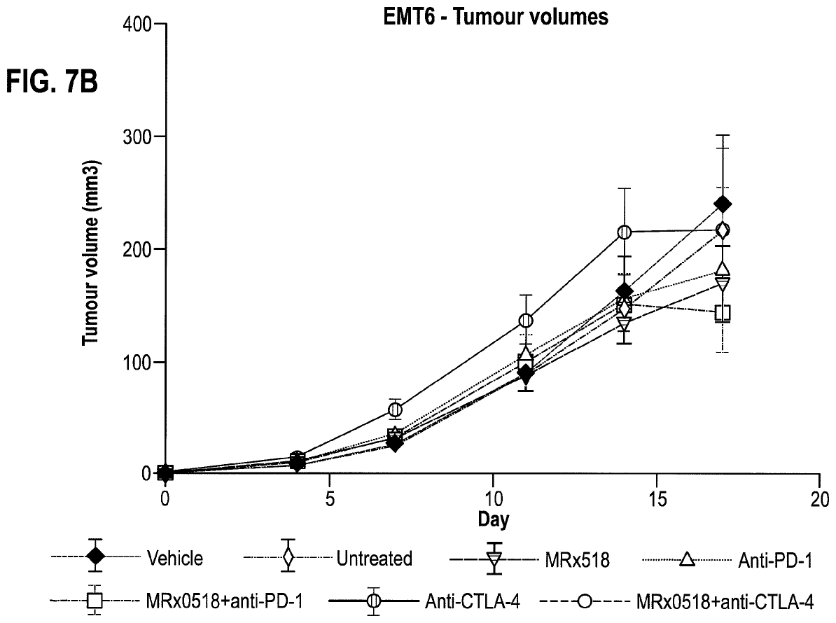

At termination, batch 3 was split into 2 cohorts, due to termination and FACS analyses schedules; these were staggered over 1 day: D24/D25. Therefore, every cohort of animals had 5 animals per treatment group (4 animals from cage one and one animal from cage 2). Based on the ethical criteria, if the tumor volume were higher than 1500 mm³, the selection of the animals to be sacrifice on D24 and D25 is based on tumor volume instead of the cage. The experimental design is depicted in FIG. 7A and summarized below:

1) Batch 1 (groups 1, 2 and 3) started treatment on D0 and was culled at D14 (10 animals form groups 1 to 3). These did not receive tumor cells and constituted the baseline group.

2) Batch 2 (group 1, 2 and 3) started treatment on D-14 and was culled at D7 (10 animals form groups 1 to 3).

3) Batch 3 (groups 1 to 7) started treatment on D-14 and was culled at D24/25 (10 animals form groups 1 to 7). The treatment of anti PD-1 and Anti CTLA-4 started on D10.

On day 0 (D0) all mice of batches 2 and 3 (termination at day 7 and 24/25, respectively) were engrafted with EMT-6 tumour cells by a subcutaneous injection of 1×10⁶ EMT-6 cells in 200 μL RPMI 1640 into the right flank (the 30 mice from batch 1, that were sacrificed on D14, did not receive the tumour injection). The mice were treated according to the following treatment schedule groups (TW×2=twice a week):

| | | | | | Treatment |
|---|---|---|---|---|---|
| Group | No. Animals | Treatment | Dose | Route | Schedule |
| 1 | 30 = | Untreated (+ | — | — | — |
| | 10 batch 1 | Tumour) | | | |
| | 10 batch 2 | | | | |
| | 10 batch 3 | | | | |
| 2 | 30 = | Vehicle (YCFA) | — | PO | Daily-14 to D0 |
| | 10 batch 1 | | | | Daily-14 to D7 |
| | 10 batch 2 | | | | Daily-14 to D24/25 |
| | 10 batch 3 | | | | |
| 3 | 30 = | MRX518 (grown | 2 × 10⁸ | PO | Daily-14 to D0 |
| | 10 batch 1 | from gly stock) in | | | Daily-14 to D7 |
| | 10 batch 2 | YCFA | | | Daily-14 to D24/25 |
| | 10 batch 3 | | | | |
| 4 | 10 batch 3 | Anti-PD-1 + | 10 mg/kg | IP + PO | TW × 2 from D10 |
| | | YCFA | | | YCFA Daily-14 to |
| | | | | | D24/25 |
| 5 | 10 batch 3 | Anti-PD-1 + | 10 mg/kg + | IP + PO | TW × 2 from D10 |
| | | MRX518 | 2 × 10⁸ bacteria | | Bacteria Daily-14 |
| | | | | | to D24/25 |
| 6 | 10 batch 3 | Anti-CTLA-4 + | 10 mg/kg | IP + PO | TW × 2 from D10 |
| | | YCFA | | | YCFA Daily-14 to |
| | | | | | D24/25 |
| 7 | 10 batch 3 | Anti- CTLA-4 + | 10 mg/kg + | IP + PO | TW × 2 from D10 |
| | | MRX518 | 2 × 10⁸ bacteria | | Bacteria Daily-14 |
| | | | | | to D24/25 |

The following samples are collected throughout the experiment:
1. Feces (only for batch 3)—At three time points during the study (D-15, D-1 and D22) faecal samples were collected from eight identical mice per group (the equivalent of 80-100 mg or 6-7 pellets per mouse, but at least 3 faecal pellets), snap frozen and stored at −80° C.
2. Blood—At the time of termination of the mice (D14 for batch1, D7 for batch 2 and D25 for batch 3), approximately 1 mL of intracardiac blood was collected from each animal into an EDTA tube in terminal procedures under deep gas anesthesia. The blood was centrifuged to obtain plasma, and the plasma stored at −80° C.
3. Tumour and spleen—The tumour (on D and D24/D25) and the spleens (on D7, D14 and D24/D25) from all mice were collected. The tumour immune infiltrate cells in the tumour samples were quantified by FACS analysis as described below.
4. Mesenteric lymph nodes—On D7, D14 and D24/D25 mesenteric lymph nodes from all animals per groups and per time point were collected and snap frozen at −80° C.
5. Intestine—At the time of euthanasia (D7, D14 and D24/D25), several sections of the intestines from all mice per group and per timing were collected and dissected. The caecal content was harvested as well.

FACS Analysis

For analysis of tumor cells, tumors from all mice per groups and per timing were collected at time of termination (on D7 and D24/25). All the tumors were collected in HBSS culture medium. The tumor immune infiltrate cells were quantified by FACS analysis from each collected sample. Briefly, the collected samples were processed by mechanic dissociation and prepared in 100 μL staining buffer (PBS, 0.2% BSA, 0.02% NaN₃). Then the antibodies directed against the chosen markers were added, according to the procedure described by the supplier for each antibody. All the antibodies except FoxP3 were for surface labeling and FoxP3 for intracellular labeling. The antibodies used for FACS analysis are listed in the tables below:

Panel 1: panel T cells viability, CD45, CD3, CD4, CD8, CD25, FOXP3, PD1, B220

| Reference | Specificity and fluorochrome | | Isotype and specificity | | Provider |
|---|---|---|---|---|---|
| 553052 | CD4 | PerCP | mouse | IgG2ak | BD biosciences |
| 553933 | IgG2a | PerCP | — | IgG2ak | BD biosciences |
| 562600 | CD3 | BV421 | mouse | IgG1k | BD biosciences |
| 562601 | IgG1 | BV421 | — | IgG1k | BD biosciences |
| 130-110-665 | CD45 | Viogreen | mouse | REA737 | Miltenyi Biotec |
| 130-104-624 | REA CTL universal | VioGreen | — | REA293 | Miltenyi Biotec |
| 563061 | CD25 | BV605 | mouse | IgG1, λ | BD biosciences |
| 562987 | IgG1 | BV605 | — | IgG1λ | BD biosciences |
| 130-111-601 | FoxP3** | APC | mouse | REA | Miltenyi Biotec |
| 130-104-615 | REA Control (1)** | APC | — | REA/hIgG1 | Miltenyi Biotec |
| 564997 | Fixable Viability Stain 700 | eq AF700 | — | — | BD biosciences |
| 130-109-250 | CD8a | APC-Vio770 | mouse | REA | Miltenyi Biotec |
| 130-104-634 | REA | APC-Vio770 | — | REA | Miltenyi Biotec |
| 130-111-800 | CD279 (=PD1) | PE | mouse | REA802 | Miltenyi Biotec |
| 130-104-628 | REA CTL universal | PE | — | REA293 | Miltenyi Biotec |
| 130-110-845 | CD45R (B220) | FITC | mouse | REA755 | Miltenyi Biotec |
| 130-104-626 | REA CTL universal | FITC | — | REA293 | Miltenyi Biotec |

Panel 2 tumor associated macrophages (TAM): viability, CD45, CD3, CD11b, Ly6C, F4/80, CD68,
CD80, CD206, MHCII

| Reference | Specificity and fluorochrome | | Isotype and specificity | | Provider |
|---|---|---|---|---|---|
| 141704 | CD206 | FITC | mouse | IgG2a | biolegend |
| 553929 | IgG2a | FITC | — | IgG2ak | BD |
| | | | | | biosciences |
| 130-116- | CD80 | PE | mouse | REA | Miltenyi |
| 396 | | | | | Biotec |
| 130-104- | REA CTL universal | PE | — | REA/hIgG1 | Miltenyi |
| 628 | | | | | Biotec |
| 130-109- | CD11b | PerCP- | mouse- | REA | Miltenyi |
| 289 | | Vio700 | human | | Biotec |
| 130-104- | REA Control (S) | PerCP | — | REA | Miltenyi |
| 620 | | Vio700 | | | Biotec |
| 130-116- | CD3 | PE-Vio770 | mouse | REA/hIgG1 | Miltenyi |
| 530 | | | | | Biotec |
| 130-104- | REA CTL universal | PE-Vio770 | — | REA/hIgG1 | Miltenyi |
| 632 | | | | | Biotec |
| 130-112- | CD68* | Vioblue | mouse | REA | Miltenyi |
| 861 | | | | | Biotec |
| 130-104- | REA CTL universal* | VioBlue | — | REA/hIgG1 | Miltenyi |
| 625 | | | | | Biotec |
| 130-102- | CD45 | Viogreen | mouse | IgG2b | Miltenyi |
| 412 | | | | | Biotec |
| 130-102- | IgG2b | VioGreen | — | IgG2b | Miltenyi |
| 659 | | | | | Biotec |
| 565694 | Fixable Viability Stain | eq BV605 | — | — | BD |
| | 575V | | | | biosciences |
| 130-102- | F4/80/EMR1 | APC | mouse | REA | Miltenyi |
| 379 | | | | | Biotec |
| 130-104- | REA CTL universal | APC | — | REA | Miltenyi |
| 630 | | | | | Biotec |
| 130-112- | MHCII | APC vio770 | mouse | REA/hIgG1 | Miltenyi |
| 233 | | | | | Biotec |
| 130-104- | REA CTL universal | APC-Vio770 | — | REA/hIgG1 | Miltenyi |
| 634 | | | | | Biotec |

The mixture was incubated for 20 to 30 minutes at room temperature in the dark, washed, and re-suspended in 200 μL staining buffer. All samples were stored on ice and protected from light until FACS analysis. Tumor samples were also processed with control isotype antibodies. The stained cells were analyzed with a CyFlow® space flow cytometer (LSR II, BD Biosciences) equipped with 3 excitation lasers at wavelengths 405, 488 and 633 nm.

For analysis of intestine samples, the small intestine and the colon of all mice per groups and per timing was collected at the time of termination (on D7, D14 and D24/25). All the fresh tissues were collected in HBSS culture medium. The immune cells in the lamina propria were quantified by FACS analysis from each collected sample. The samples were processed as the tumor samples. The antibodies used for FACS analysis are those of panel 1 listed above and those listed in the table below (subsequent incubation of samples and analysis were as described above):

Panel 3: intestinal DCs: viability, CD45, CD3, CD11b, CD11c, MHC II, CD103

| Reference | Specificity and fluorochrome | | Isotype and specificity | | Provider |
|---|---|---|---|---|---|
| 130-109- | CD 11b | PerCP- | mouse- | REA | Miltenyi |
| 289 | | Vio700 | human | | Biotec |
| 130-104- | REA Control (S) | PerCP Vio700 | — | REA | Miltenyi |
| 620 | | | | | Biotec |
| 130-116- | CD3 | PE-Vio770 | mouse | REA/hIgG1 | Miltenyi |
| 530 | | | | | Biotec |
| 130-104- | REA CTL universal | PE-Vio770 | — | REA/hIgG1 | Miltenyi |
| 632 | | | | | Biotec |
| 560583 | CD 11c | AlexaFluor | mouse | IgGI | BD |
| | | 700 | | | bioscience |
| 560555 | IgGI | AlexaFluor | — | IgG2 | BD |
| | | 700 | | | bioscience |
| 130-102- | CD45 | Viogreen | mouse | IgG2b | Miltenyi |
| 412 | | | | | Biotec |
| 130-102- | IgG2b | VioGreen | — | IgG2b | Miltenyi |
| 659 | | | | | Biotec |
| 565694 | Fixable Viability Stain | eq BV605 | — | — | BD |
| | 575V | | | | biosciences |
| 130-108- | CD 103 | APC | mouse | REA | Miltenyi |
| 184 | | | | | Biotec |
| 130-104- | REA CTL universal | APC | — | REA/hIgG1 | Miltenyi |
| 630 | | | | | Biotec |
| 130-112- | MHCII | APC vio770 | mouse | REA/hIgG1 | Miltenyi |
| 233 | | | | | Biotec |
| 130-104- | REA CTL universal | APC-Vio770 | — | REA/hIgG1 | Miltenyi |
| 634 | | | | | Biotec |
| 130-102- | F4/80/EMR1 | FITC | mouse | REA126 | Miltenyi |
| 327 | | | | | Biotec |
| 130-104- | REA CTL universal | FITC | — | REA293 | Miltenyi |
| 626 | | | | | Biotec |

For analysis of spleen samples, the spleen of all mice per groups and per timing was collected at the time of termination (on D7, D14 and D24/25). All the spleens were collected in complete RPMI culture medium (10% dFBS, Penicillin/streptomycin 1%, 2 mM L-glutamine and 55 μM 2-mercaptoethanol). The tumor immune infiltrate cells were quantified by FACS analysis from each collected sample after stimulation for 72 h with CD3 and CD28. Procedure: Splenocytes were cultured with either one of two stimulations (CD3/CD28, heat-killed MRx0518) and one negative control. There was a ratio of 1:1 between the heat-killed MRx0518 and the splenocytes per well. There was 1×106 bacterial cells provided in 20 μl of the heat-killed MRx0518 sample. The antibodies directed against the markers of panel 1 above were added to cell pellets from each treatment, according to the procedure described by the supplier for each antibody. Subsequent incubation of samples and analysis were performed as described above.

Animal Monitoring

The viability and behaviour of the animals was recorded every day. Body weights were measured twice a week. The length and width of the tumour was measured twice a week with callipers and the volume of the tumour was estimated by the following formula:

$$Tumour volume = \frac{{Width}^{2} \times Length}{2}$$

The treatment efficacy was assessed in terms of the effects of the test substance on the tumour volumes of treated animals relative to control animals. The following evaluation criteria of antitumor efficacy were determined using Vivo Manager® software (Biosystemes, Couternon, France):
1. Individual and/or mean (or median) tumour volumes. Mean tumour volumes of groups 1 to 7 are depicted in FIG. 7B.
2. Tumour doubling time (DT).
3. Tumour growth inhibition (T/C %) defined as the ratio of the median tumor volumes of treated versus control group, calculated as follows (Dx=Day of measurement):

$$T / C \% = \frac{Median tumour valume of treated group at {D}_{x}}{Median tumour volume of vehicle treated group at {D}_{x}} \times 100$$

The optimal value is the minimal T/C % ratio reflecting the maximal tumour growth inhibition achieved. The effective criteria for the T/C % ratio, according to NCI standards, is 42%.
4. Relative tumour volume (RTV) curves of test and control groups, where the RTV is calculated as follows (Dx=Day of measurement; DR=Day of randomization):

$$RTV = \frac{TV at {D}_{X}}{TV at {D}_{R}}$$

5. Volume V and time to reach V are calculated. Volume V is defined as a target volume deduced from experimental data and chosen in exponential phase of tumour growth. For each tumour, the closest tumour volume to the target volume V is selected in tumour volume measurements. The value of this volume V and the time for the tumour to reach this volume are recorded. For each group, the mean of the tumour volumes V and the mean of the times to reach this volume are calculated.

### Example 7—CD8 Proliferation Assessment

To investigate the immunostimulatory effects of MRX518 and pembrolizumab, an in vitro assessment of the impact on CD8+ cell proliferation of MRX518 and an anti PD-1 checkpoint inhibitor in combination was conducted.

Peripheral blood mononuclear cells (PBMCs, cryopreserved from Stemcell Technologies, catalogue number: 70025), were removed from liquid nitrogen and allowed to rest overnight in a flask. A 96-well plate was coated with CD3 antibody (ThermoFisher CD3 Monoclonal Antibody (OKT3), 0.3 μg/ml) as one half of a mitogenic combination. Following the resting period, the PBMCs were counted and stained with fluorescent cell tracer (CellTrace™ Far Red Cell Proliferation Kit).

Ten sets of cells were prepared in this way. To nine of those sets, anti PD-1 antibody was added (from Miltenyi Biotech CD279 (PD1) pure functional grade, 10 μs/ml). No anti PD-1 antibody was added to the additional set, which served as a control set (referred to as Cell Set 1 in the below table). All cell sets were then incubated for 1.5 hours.

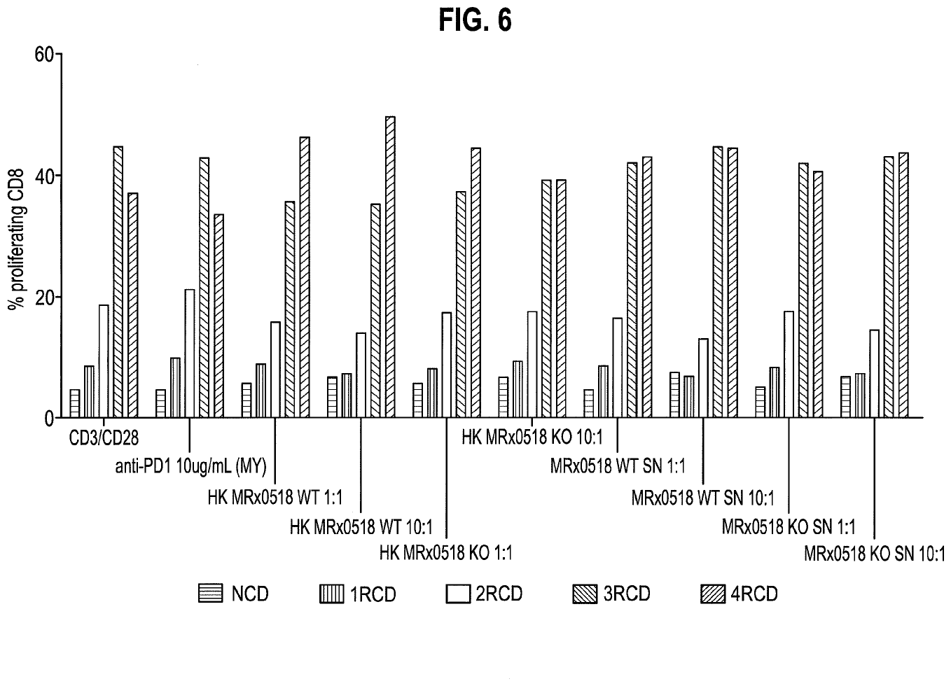

Following the incubation period, bacterial test components were added to Cell Sets 3 to 10 as shown in the following table:

| Cell | | Acronym as presented in |
|---|---|---|
| Set | Bacterial Component | Figure 6 |
| 1 | None, anti PD-1 free control | CD3/CD28 |
| 2 | None, anti PD-1 control | anti-PDl 10 ug/ml (MY) |
| 3 | Heat Killed MRX518 at a ratio of 1:1 * | HK MRx0518 WT 1:1 |
| 4 | Heat Killed MRX518 at a ratio of 10:1* | HK MRx0518 WT 10:1 |
| 5 | Heat Killed MRX518 with flagellin knockout** at a ratio | HK MRx0518 KO 1:1 |
| | of 1:1* | |
| 6 | Heat Killed MRX518 with flagellin knockout** at a | HK MRx0518 KO 10:1 |
| | ratio of 10:1* | |
| 7 | MRX518 supernatant at a ratio of 1:1 * * * | HKMRx0518 WT SN 1:1 |
| 8 | MRX518 supernatant at a ratio of 10:1 * * * | HK MRx0518 WT SN 10:1 |
| 9 | MRX518 flagellin knockout supernatant** at a ratio of | HK MRx0518 KO SN 1:1 |
| | 1-1*** | |
| 10 | MRX518 flagellin knockout supernatant** at a ratio of | HK MRx0518 KO SN 10:1 |
| | 10:1*** | |

*Ratio of MRX518 cells:PBMC cells
**A mutant of MRX518 engineered to have a disrupted flagellar assembly was tested. The flagellin is understood by the inventors to contribute to the immunostimulatory effect of MRX518
***For the 1:1 Multiplicity Of Infection (MOI), the supernatant was taken from the same number of bacteria as the number of PBMCs treated with the supernatant. For the MOI of 10:1, the supernatant was taken from a highly concentrated bacterial culture, but the precise number of bacteria with respect to the PBMCs was not measured.

Following the addition of the bacterial test components, a CD28 antibody (Thermofisher CD28 Monoclonal Antibody (CD28.2), 1 μg/ml) was added to each of the cell sets as the other half of the mitogenic combination, to trigger proliferation. PDL-1 (R&D Systems, Recombinant Human PD-L1/B7-H1 Fc Chimera, 10 μg/ml) was then added to each cell set.

The cell sets were then incubated for 5 days (37° C., 5% CO₂). Following the incubation, the cells were harvested and analysed by FACS according to cellular fluorescence imparted by the cell tracer, providing an indication of the number of cell divisions that had occurred in the incubation period. The results showing the percentages of cells grouped into the number of divisions (from no cell division (NCD) to 4 cell divisions (4RCD)) are shown in FIG. 6.

| Sequences | |
|---|---|
| (Enterococcus gallinarum 16S rRNA gene-AF039900) | |
| SEQ ID NO: 1 | |
| 1 | taatacatgc aagtcgaacg ctttttcttt caccggagct tgctccaccg aaagaaaaag |
| 61 | agtggcgaac gggtgagtaa cacgtgggta acctgcccat cagaagggga taacacttgg |
| 121 | aaacaggtgc taataccgta taacactatt ttccgcatgg aagaaagttg aaaggcgctt |
| 181 | ttgcgtcact gatggatgga cccgcggtgc attagctagt tggtgaggta acggctcacc |
| 241 | aaggccacga tgcatagccg acctgagagg gtgatcggcc acactgggac tgagacacgg |
| 301 | cccagactcc tacgggaggc agcagtaggg aatcttcggc aatggacgaa agtctgaccg |
| 361 | agcaacgccg cgtgagtgaa gaaggttttc ggatcgtaaa actctgttgt tagagaagaa |
| 421 | caaggatgag agtagaacgt tcatcccttg acggtatcta accagaaagc cacggctaac |
| 481 | tacgtgccag cagccgcggt aatacgtagg tggcaagcgt tgtccggatt tattgggcgt |
| 541 | aaagcgagcg caggcggttt cttaagtctg atgtgaaagc ccccggctca accggggagg |
| 601 | gtcattggaa actgggagac ttgagtgcag aagaggagag tggaattcca tgtgtagcgg |
| 661 | tgaaatgcgt agatatatgg aggaacacca gtggcgaagg cggctctctg gtctgtaact |
| 721 | gacgctgagg ctcgaaagcg tggggagcga acaggattag ataccctggt agtccacgcc |
| 781 | gtaaacgatg agtgctaagt gttggagggt ttccgccctt cagtgctgca gcaaacgcat |
| 841 | taagcactcc gcctggggag tacgaccgca aggttgaaac tcaaaggaat tgacgggggc |
| 901 | ccgcacaagc ggtggagcat gtggtttaat tcgaagcaac gcgaagaacc ttaccaggtc |
| 961 | ttgacatcct ttgaccactc tagagataga gcttcccctt cgggggcaaa gtgacaggtg |
| 1021 | gtgcatggtt gtcgtcagct cgtgtcgtga gatgttgggt taagtcccgc aacgagcgca |
| 1081 | acccttattg ttagttgcca tcatttagtt gggcactcta gcgagactgc cggtgacaaa |
| 1141 | ccggaggaag gtggggatga cgtcaaatca tcatgcccct tatgacctgg gctacacacg |
| 1201 | tgctacaatg ggaagtacaa cgagttgcga agtcgcgagg ctaagctaat ctcttaaagc |
| 1261 | ttctctcagt tcggattgta ggctgcaact cgcctacatg aagccggaat cgctagtaat |
| 1321 | cgcggatcag cacgccgcgg tgaatacgtt cccgggcctt gtacacaccg cccgtcacac |
| 1381 | cacgagagtt tgtaacaccc gaagtcggtg aggtaacctt tttggagcca gccgcctaag |
| 1441 | gtgggataga tgattggggt gaagtcgtaa caaggtagcc gtatcggaag gtgcggctgg |
| 1501 | atcacc |

(consensus 16S rRNA sequence for Enterococcus gallinarum strain MRX518)
SEQ ID NO: 2
TGCTATACATGCAGTCGAACGCTTTTTCTTTCACCGGAGCTTGCTCCACCGAAAGAAAAAGAGTGGCGAACGGGTGA
GTAACACGTGGGTAACCTGCCCATCAGAAGGGGATAACACTTGGAAACAGGTGCTAATACCGTATAACACTATTTTC
CGCATGGAAGAAAGTTGAAAGGCGCTTTTGCGTCACTGATGGATGGACCCGCGGTGCATTAGCTAGTTGGTGAGGTA
ACGGCTCACCAAGGCCACGATGCATAGCCGACCTGAGAGGGTGATCGGCCACACTGGGACTGAGACACGGCCCAGAC
TCCTACGGGAGGCAGCAGTAGGGAATCTTCGGCAATGGACGAAAGTCTGACCGAGCAACGCCGCGTGAGTGAAGAAG
GTTTTCGGATCGTAAAACTCTGTTGTTAGAGAAGAACAAGGATGAGAGTAGAACGTTCATCCCTTGACGGTATCTAA
CCAGAAAGCCACGGCTAACTACGTGCCAGCAGCCGCGGTAATACGTAGGTGGCAAGCGTTGTCCGGATTTATTGGGC
GTAAAGCGAGCGCAGGCGGTTTCTTAAGTCTGATGTGAAAGCCCCCGGCTCAACCGGGGAGGGTCATTGGAAACTGG
GAGACTTGAGTGCAGAAGAGGAGAGTGGAATTCCATGTGTAGCGGTGAAATGCGTAGATATATGGAGGAACACCAGT
GGCGAAGGCGGCTCTCTGGTCTGTAACTGACGCTGAGGCTCGAAAGCGTGGGGAGCGAACAGGATTAGATACCCTGG
TAGTCCACGCCGTAAACGATGAGTGCTAAGTGTTGGAGGGTTTCCGCCCTTCAGTGCTGCAGCAAACGCATTAAGCA
CTCCGCCTGGGGAGTACGACCGCAAGGTTGAAACTCAAAGGAATTGACGGGGGCCCGCACAAGCGGTGGAGCATGTG
GTTTAATTCGAAGCAACGCGAAGAACCTTACCAGGTCTTGACATCCTTTGACCACTCTAGAGATAGAGCTTCCCCTT
CGGGGGCAAAGTGACAGGTGGTGCATGGTTGTCGTCAGCTCGTGTCGTGAGATGTTGGGTTAAGTCCCGCAACGAGC
GCAACCCTTATTGTTAGTTGCCATCATTTAGTTGGGCACTCTAGCGAGACTGCCGGTGACAAACCGGAGGAAGGTGG
GGATGACGTCAAATCATCATGCCCCTTATGACCTGGGCTACACACGTGCTACAATGGGAAGTACAACGAGTTGCGAA
GTCGCGAGGCTAAGCTAATCTCTTAAAGCTTCTCTCAGTTCGGATTGTAGGCTGCAACTCGCCTACATGAAGCCGGA
ATCGCTAGTAATCGCGGATCAGCACGCCGCGGTGAATACGTTCCCGGGCCTTGTACACACCGCCCGTCACACCACGA
GAGTTTGTAACACCCGAAGTCGGTGAGGTAACCTTTTTGGAGCCAGCCGCCTAAGGTG

### REFERENCES

[1] Spor et al. (2011) Nat Rev Microbiol. 9(4):279-90.
[2] Eckburg et al. (2005) Science. 10; 308(5728):1635-8.
[3] Macpherson et al. (2001) Microbes Infect. 3(12):1021-35
[4] Macpherson et al. (2002) Cell Mol Life Sci. 59(12):2088-96.
[5] Mazmanian et al. (2005) Cell 15; 122(1):107-18.
[6] Frank et al. (2007) PNAS 104(34):13780-5.
[7] Scanlan et al. (2006) J Clin Microbiol. 44(11):3980-8.
[8] Kang et al. (2010) Inflamm Bowel Dis. 16(12):2034-42.
[9] Machiels et al. (2013) Gut. 63(8):1275-83.
[10] WO 2013/050792
[11] WO 03/046580
[12] WO 2013/008039
[13] WO 2014/167338
[14] Goldin and Gorbach (2008) Clin Infect Dis. 46 Suppl 2:S96-100.
[15] Azad et al. (2013) BMJ. 347:f6471.
[16] Strickertsson et al. (2014) Genes. 5(3): 726-738.
[17] Collins et al. (1984) Int J Syst Evol Microbiol. 34: 220-223.
[18] Masco et al. (2003) Systematic and Applied Microbiology, 26:557-563.
[19] Srůtková et al. (2011) J. Microbiol. Methods, 87(1): 10-6.
[20] Haabeth et al. (2012) Oncolmmunology 1(1):1146-1152.
[21] Lejeune et al. (2006) Cancer Immun. 6:6
[22] Pace et al. (1983) PNAS. 80:8782-6.
[23] Sgadari et al. (1996) PNAS. 93:13791-6.
[24] Arenberg et al. (1996) J. Exp. Med. 184:981-92.
[25] Sgadari et al. (1997) Blood. 89:2635-43.
[26] Miyamoto-Shinohara et al. (2008) J. Gen. Appl. Microbiol., 54, 9-24.
[27] Cryopreservation and Freeze-Drying Protocols, ed. by Day and McLellan, Humana Press.
[28] Leslie et al. (1995) Appl. Environ. Microbiol. 61, 3592-3597.
[29] Mitropoulou et al. (2013) J Nutr Metab. (2013) 716861.
[30] Kailasapathy et al. (2002) Curr Issues Intest Microbiol. 3(2):39-48.
[31] Handbook of Pharmaceutical Excipients, 2nd Edition, (1994), Edited by A Wade and P J Weller
[32] Remington's Pharmaceutical Sciences, Mack Publishing Co. (A. R. Gennaro edit. 1985)
[33] US 2016/0067188
[34] Handbook of Microbiological Media, Fourth Edition (2010) Ronald Atlas, CRC Press.
[35] Maintaining Cultures for Biotechnology and Industry (1996) Jennie C. Hunter-Cevera, Academic Press
[36] Strobel (2009) Methods Mol Biol. 581:247-61.
[37] Gennaro (2000) Remington: The Science and Practice of Pharmacy. 20th edition, ISBN: 0683306472.
[38] Molecular Biology Techniques: An Intensive Laboratory Course, (Ream et al., eds., 1998, Academic Press).
[39] Methods In Enzymology (S. Colowick and N. Kaplan, eds., Academic Press, Inc.)
[40] Handbook of Experimental Immunology, Vols. I-IV (D. M. Weir and C. C. Blackwell, eds, 1986, Blackwell Scientific Publications)
[41] Sambrook et al. (2001) Molecular Cloning: A Laboratory Manual, 3rd edition (Cold Spring Harbor Laboratory Press).
[42] Handbook of Surface and Colloidal Chemistry (Birdi, K. S. ed., CRC Press, 1997)
[43] Ausubel et al. (eds) (2002) Short protocols in molecular biology, 5th edition (Current Protocols).
[44] PCR (Introduction to Biotechniques Series), 2nd ed. (Newton & Graham eds., 1997, Springer Verlag)
[45] Current Protocols in Molecular Biology (F. M. Ausubel et al., eds., 1987) Supplement 30
[46] Smith & Waterman (1981) Adv. Appl. Math. 2: 482-489.
[47] Rockwell et al., (1972) J Natl Cancer Inst. 49:735-49.
[48] Bertram and Jarvik (1980) Cancer Lett. 11:63-73.
[49] Darlington (1987) Meth Enzymol. 151:19-38.
[50] Principe d'éthique de l'expérimentation animale, Directive no 2010/63 CEE 22 Sep. 2010, Décêr& no 2013-118 1 Feb. 2013.
[51] Guide for the Care and Use of Laboratory Animals: Eighth Edition. The National Academies Press; 2011
[52] Simpson-Herren and Lloyd (1970) Cancer Chemother Rep. 54:143-74.
[53] Workman et al. (2010) Br. J. Cancer. 102:1555-77.
[54] WO 2017/085520

1. A therapeutic combination for use in a method of treating or preventing cancer in a subject, wherein said therapeutic combination comprises:
(a) a composition comprising a bacterial strain of the species Enterococcus gallinarum; and
(b) pembrolizumab.

2.-24. (canceled)
